(19) United States
(12) Patent Application Publication
(10) Pub. No.: US 2004/0259857 A1
(43) Pub. Date: Dec. 23, 2004

(54) Process for the preparation of nonpeptide substituted spirobenzoazepine derivatives

(76) Inventors: Xiaohu Deng, San Diego, CA (US); Birdella Kenney, North Wales, PA (US); Jimmy T. Liang, San Diego, CA (US); Neelakandha Mani, San Diego, CA (US); Frank J. Villani, Perkasie, PA (US); Fan Zhang-Plasket, Willow Grove, PA (US); Hua Zhong, Maple Glen, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/869,615

(22) Filed: Jun. 16, 2004

Related U.S. Application Data
(60) Provisional application No. 60/479,515, filed on Jun. 17, 2003.

(51) Int. Cl. A61K 31/55; C07D 491/10; C07D 487/10

(52) U.S. Cl. 514/217; 540/543

(57) ABSTRACT

Novel spirobenzoazepine compounds, novel processes for the preparation of nonpeptide substituted spirobenzoazepine derivatives, and novel processes for the preparation of intermediates in the preparation of such derivatives. Novel intermediates in the preparation of nonpeptide substituted spirobenzoazepine derivatives.

### FIELD OF THE INVENTION

The present invention is directed to a novel process for the preparation of nonpeptide substituted spirobenzoazepine derivatives and to novel processes for the preparation of intermediates in the preparation of said derivatives. The present invention is further directed to novel intermediates in the preparation of nonpeptide substituted spirobenzoazepine derivatives.

### BACKGROUND OF THE INVENTION

The present invention is directed to a novel process for the preparation of nonpeptide substituted spirobenzoazepine derivatives useful for treating and/or preventing conditions involving increased vascular resistance and cardiac insufficiency. More particularly the nonpeptide substituted spirobenzoazepine derivatives are useful in the treatment and/or prevention of disorders such as aggression, obsessive-compulsive disorders, hypertension, dysmenorrhea, congestive heart failure/cardiac insufficiency, coronary vasospasm, cardiac ischemia, liver cirrhosis, renal vasospasm, renal failure, edema, ischemia, stroke, thrombosis, water retention, nephritic syndrome and central nervous system injuries.

Chen et al., in PCT publication WO 02/02531 disclose a process for the preparation of nonpeptide substituted spirobenzoazepines. However, this process requires the use of an explosive reagent, cryogenic temperature and chromatographic purifications, and is therefore not desirable for commercial preparation of said compounds.

### SUMMARY OF THE INVENTION

The present invention is directed to a process for the preparation of compounds of formula (I)

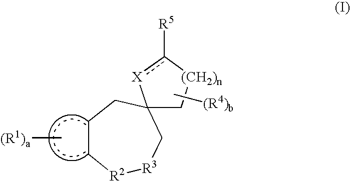

wherein

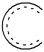

is selected from the group consisting of aryl and heteroaryl; provided that the heteroaryl group does not contain a nitrogen atom;

a is an integer from 1 to 3;

R¹ is selected from the group consisting of hydrogen, halogen, hydroxy, alkoxy, phenyl, substituted phenyl, alkylthio, arylthio, alkyl-sulfoxide, aryl-sulfoxide, alkyl-sulfone and aryl-sulfone;

—R²—R³— is selected from the group consisting of

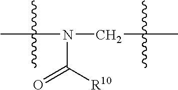

and

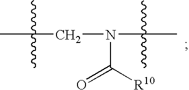

R¹⁰ is selected from the group consisting of alkyl, substituted alkyl, phenyl, substituted phenyl, heteroaryl, substituted heteroaryl and —(B)₀₋₁-G-(E)₀₋₁-(W)₁₋₃;

wherein B is selected from (CH₂)₁₋₃, NH or O;

G is selected from aryl, substituted aryl, heteroaryl or substituted heteroaryl;

E is selected from —O—, —S—, —NH—, —(CH₂)₀₋₃—N(R¹¹)C(O)— or —(CH₂)₀₋₃—C(O)NR¹¹; wherein R¹¹ is selected from the group consisting of hydrogen, alkyl and substituted alkyl;

each W is independently selected from hydrogen, alkyl, substituted alkyl, amino, substituted amino, alkylthiophenyl, alkyl-sulfoxidephenyl, aryl, substituted aryl, heteroaryl or substituted heteroaryl;

X is selected from the group consisting of CH, CH₂, CHOH and C(O);

[symbol] represents a single or double bond;

provided that when R¹ is iodine, bromine, alkylthio, arylthio, alkyl-sulfone or aryl-sulfone, then [symbol] is a double bond;

n is an integer from 1 to 3;

b is an integer from 1 to 2;

R⁴ is selected from the group consisting of hydrogen, alkyl, substituted alkyl, phenyl and substituted phenyl;

R⁵ is selected from the group consisting of hydrogen, alkyl, substituted alkyl, aldehyde, carboxyl, alkoxycarbonyl, substituted alkoxycarbonyl, —(CH₂)ₖNZ¹Z² and —C(O)NZ¹Z²;

wherein k is an integer from 1 to 4;

Z¹ and Z² are independently selected from hydrogen, alkyl, substituted alkyl, heterocyclyl, substituted heterocyclyl, aminocarbonyl or substituted aminocarbonyl;

alternatively Z¹ and Z² are taken together with the N atom to which they are bound to form a heterocyclyl, substituted heterocyclyl, heteroaryl or substituted heteroaryl;

or an optical isomer, enantiomer, diastereomer, racemate thereof, or a pharmaceutically acceptable salt thereof;

comprising

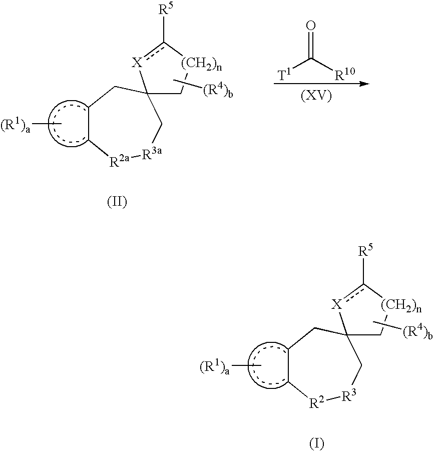

reacting a compound of formula (II) wherein —R^{2a}—R^{3a}— is selected from the group consisting of —NH—CH₂— and —CH₂—NH— with a compound of formula (XV), wherein T¹ is Cl, Br or F; in the presence of a base capable of neutralizing HT¹; in a non-alcoholic organic solvent or a mixture of a non-alcoholic organic solvent and water, to yield the corresponding compound of formula (I).

The present invention is further directed to a process for the preparation of a compound of formula (II)

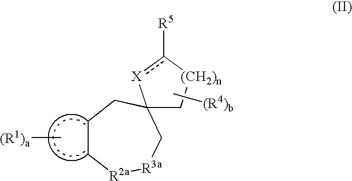

wherein

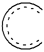

is selected from the group consisting aryl and heteroaryl; provided that the heteroaryl does not contain a nitrogen atom;

a is an integer from 1 to 3;

R¹ is selected from the group consisting of hydrogen, halogen, hydroxy, alkoxy, phenyl, substituted phenyl, alkylthio, arylthio, alkyl-sulfoxide, aryl-sulfoxide, alkyl-sulfone and aryl-sulfone;

—R^{2a}—R^{3a}— is selected from the group consisting of —NH—CH₂— and —CH₂—NH—;

X is selected from the group consisting of CH, CH₂, CHOH and C(O);

[symbol] represents a single or double bond;

provided that when R¹ is iodine, bromine, alkylthio, arylthio, alkyl-sulfone or aryl-sulfone, then [symbol] is a double bond;

n is an integer from 1 to 3;

b is an integer from 1 to 2;

R⁴ is selected from the group consisting of hydrogen, alkyl, substituted alkyl, phenyl and substituted phenyl;

R⁵ is selected from the group consisting of hydrogen, alkyl, substituted alkyl, aldehyde, carboxyl, alkoxycarbonyl, substituted alkoxycarbonyl, —(CH₂)ₖNZ¹Z² and —C(O)NZ¹Z²;

wherein k is an integer from 1 to 4;

Z¹ and Z² are independently selected from hydrogen, alkyl, substituted alkyl, heterocyclyl, substituted heterocyclyl, aminocarbonyl or substituted aminocarbonyl;

alternatively Z¹ and Z² are taken together with the N atom to which they are bound to form a heterocyclyl, substituted heterocyclyl, heteroaryl or substituted heteroaryl;

or an optical isomer, enantiomer, diastereomer, racemate thereof, or a pharmaceutically acceptable salt thereof;

comprising

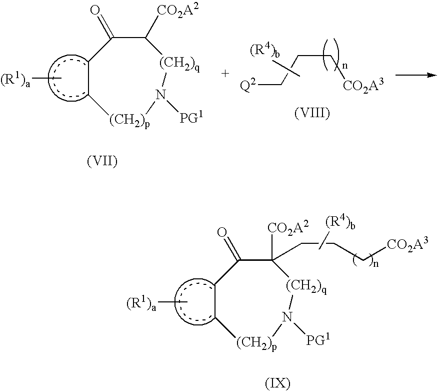

reacting a compound of formula (VII), wherein p is an integer from 0 to 1, q is an integer from 1 to 2, provided that when p is 0 then q is 2 and when p is 1 then q is 1, PG¹ is a nitrogen protecting group and A² is lower alkyl, with a compound of formula (VIII) wherein Q² is a leaving group and A³ is lower alkyl; in the presence of a base capable of deprotonating an alpha proton to the ketone on the compound of formula (VII); in an aprotic solvent, to yield the corresponding compound of formula (IX);

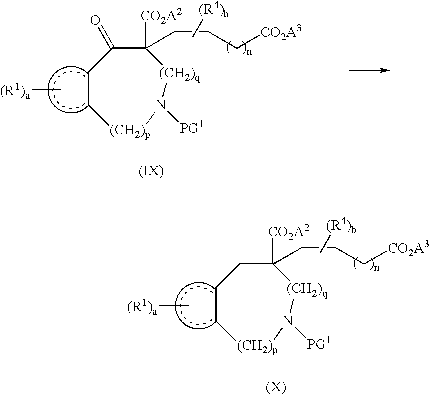

reducing the compound of formula (IX) to yield the corresponding compound of formula (X);

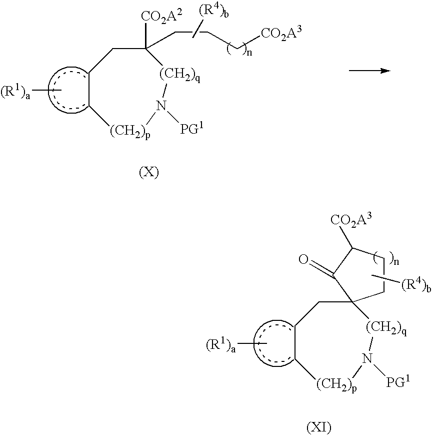

reacting the compound of formula (X) in the presence of a base capable of deprotonating an alpha proton to the CO₂A³ substituent; in an organic solvent that does not prevent the deprotonation of an alpha proton to the CO₂A³ substituent, to yield the corresponding compound of formula (XI);

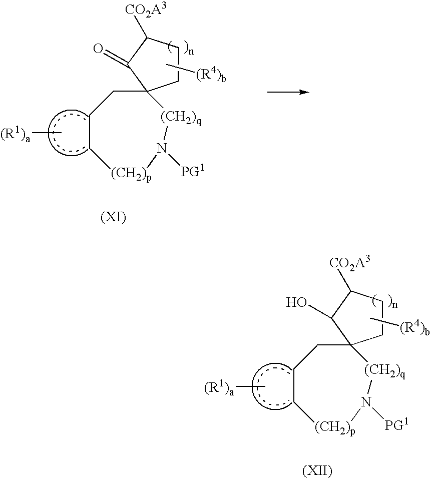

reducing the compound of formula (XI) to yield the corresponding compound of formula (XII);

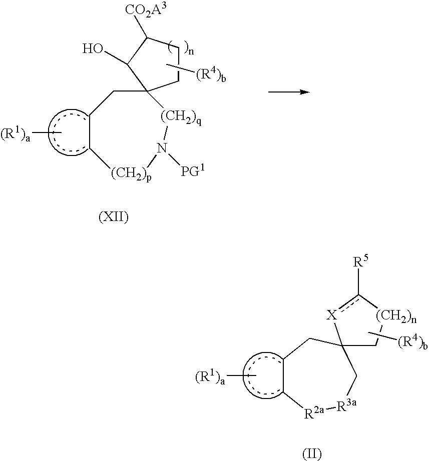

reacting the compound of formula (XII) to yield the corresponding compound of formula (II).

The present invention is further directed to a process for the preparation of compounds of formula (XVa)

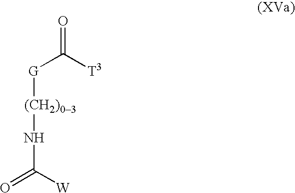

wherein

T³ is selected from the group consisting of Cl, Br and F;

G is selected from aryl, substituted aryl, heteroaryl or substituted heteroaryl;

W is selected from hydrogen, alkyl, substituted alkyl, amino, substituted amino, alkylthiophenyl, alkyl-sulfoxidephenyl, aryl, substituted aryl, heteroaryl or substituted heteroaryl;

comprising

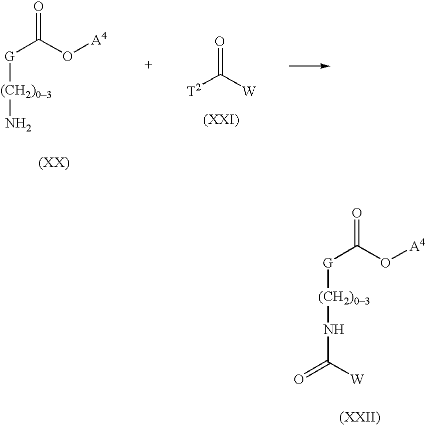

reacting a compound of formula (XX), wherein A⁴ is lower alkyl with a compound of formula (XXI) wherein T² is Cl, Br or F; in the presence of a base capable of neutralizing HT², in a non-alcoholic organic solvent or in a mixture of a non-alcoholic organic solvent and water, to yield the corresponding compound of formula (XXII);

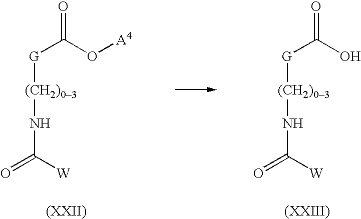

hydrolyzing the compound of formula (XXII), to yield the corresponding compound of formula (XXIII);

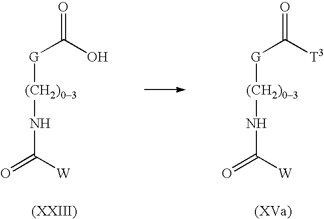

reacting the compound of formula (XXIII) with a reagent capable of converting the —CO₂H substituent to the corresponding —C(O)T³ substituent; in an inert organic solvent, to yield the corresponding compound of formula (XVa).

The present invention is further directed to a compound of formula (II)

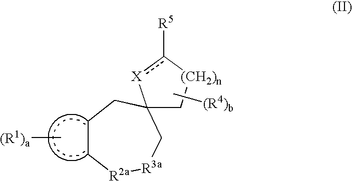

wherein

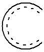

is selected from the group consisting aryl and heteroaryl; provided that the heteroaryl does not contain a nitrogen atom;

a is an integer from 1 to 3;

R¹ is selected from the group consisting of hydrogen, halogen, hydroxy, alkoxy, phenyl, substituted phenyl, alkylthio, arylthio, alkyl-sulfoxide, aryl-sulfoxide, alkyl-sulfone and aryl-sulfone;

—R^{2a}—R^{3a}— is selected from the group consisting of —NH—CH₂— and —CH₂—NH—;

X is selected from the group consisting of CH, CH₂, CHOH and C(O);

[symbol] represents a single or double bond;

provided that when R¹ is iodine, bromine, alkylthio, arylthio, alkyl-sulfone or aryl-sulfone, then is [symbol] a double bond;

n is an integer from 1 to 3;

b is an integer from 1 to 2;

R⁴ is selected from the group consisting of hydrogen, alkyl, substituted alkyl, phenyl and substituted phenyl;

R⁵ is selected from the group consisting of hydrogen, alkyl, substituted alkyl, aldehyde, carboxyl, alkoxycarbonyl, substituted alkoxycarbonyl, —(CH₂)ₖNZ¹Z² and —C(O)NZ¹Z²;

wherein k is an integer from 1 to 4;

Z¹ and Z² are independently selected from hydrogen, alkyl, substituted alkyl, heterocyclyl, substituted heterocyclyl, aminocarbonyl or substituted aminocarbonyl;

alternatively Z¹ and Z² are taken together with the N atom to which they are bound to form a heterocyclyl, substituted heterocyclyl, heteroaryl or substituted heteroaryl;

or an optical isomer, enantiomer, diastereomer, racemate thereof, or a pharmaceutically acceptable salt thereof

The present invention is further directed to a process for the preparation of (4R)-1,2,3,5-tetrahydro-spiro[4H-1-benzazepine-4,1′-[2]cyclopentene]-3′-carboxylic acid comprising reacting a racemic mixture of 1,2,3,5-tetrahydro-spiro[4H-1-benzazepine-4,1′-[2]cyclopentene]-3′-carboxylic acid with (−)-camphorsulfonic acid.

The present invention is further directed to a process for the preparation of (4S)-1,2,3,5-tetrahydro-spiro[4H-1-benzazepine-4,1′-[2]cyclopentene]-3′-carboxylic acid comprising reacting a racemic mixture of 1,2,3,5-tetrahydro-spiro[4H-1-benzazepine-4,1′-[2]cyclopentene]-3′-carboxylic acid with (+)-camphorsulfonic acid.

The present invention is further directed to novel salts of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, a compound of formula (Ia)

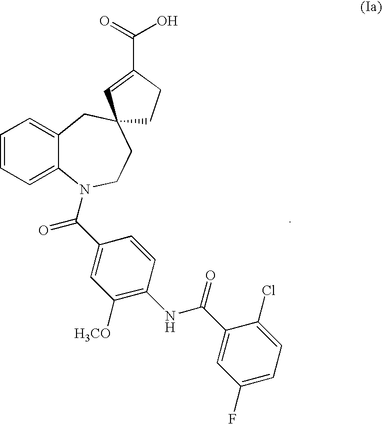

More particularly, the present invention is directed to diethylamine, pipierazine and 1-(2-hydroxyethyl)pyrrolidine salts of (4R)-1-[4-(2-chloro-5-fluorobenzoyl)amino-3-methoxybenzoyl]-1, 2, 3, 5-tetrahydro-spiro[4H-1-benzazepine-4,1′-[2]cyclopentene]-3′-carboxylic acid.

The present invention is further directed to novel processes for the preparation of the novel crystalline salts of the compounds of formula (Ia).

The present invention is further directed to a compound prepared according to any of the processes described herein.

Illustrative of the invention is a pharmaceutical composition comprising a pharmaceutically acceptable carrier and a compound prepared according to any of the processes described herein. An illustration of the invention is a pharmaceutical composition made by mixing a compound prepared according to any of the processes described herein and a pharmaceutically acceptable carrier. Illustrating the invention is a process for making a pharmaceutical composition comprising mixing a compound prepared according to any of the processes described herein and a pharmaceutically acceptable carrier.

Another example of the invention is the use of a compound prepared according to any of the processes described herein in the preparation of a medicament for treating at least one of: (a) aggression, (b) obsessive-compulsive disorders, (c) hypertension, (d) dysmenorrhea, (e) congestive heart failure/cardiac insufficiency, (f) coronary vasospasm, (g) cardiac ischemia, (h) liver cirrhosis, (i) renal vasospasm, (j) renal failure, (k) edema, (l) ischemia, (m) stroke, (n) thrombosis, (o) water retention, (p) nephritic syndrome and (q) central nervous system injuries, in a subject in need thereof.

### DETAILED DESCRIPTION OF THE INVENTION

The present invention is directed to a process for the preparation of compounds of formula (I)

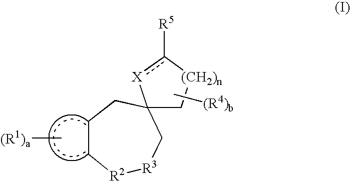

wherein

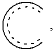

a, R¹, —R²—R³—, X, [symbol] , R⁵, n, b and R⁴ are as herein defined. The compounds of formula (I) interrupt the binding of the peptide hormone vassopressin to its receptors and are therefore useful for treating conditions involving increased vascular resistance and cardiac insufficiency. More particularly, compounds of formula (I) are useful in the treatment and/or prevention of disorders such as aggression, obsessive-compulsive disorders, hypertension, dysmenorrhea, congestive heart failure/cardiac insufficiency, coronary vasospasm, cardiac ischemia, liver cirrhosis, renal vasospasm, renal failure, edema, ischemia, stroke, thrombosis, water retention, nephritic syndrome and central nervous system injuries.

The present invention is further directed to a process for the preparation of compounds of formula (II)

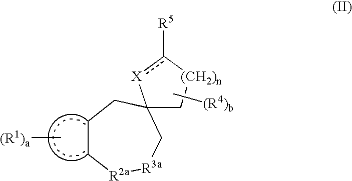

wherein

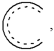

a, R¹, —R^{2a}—R^{3a}—, X, [symbol] , R⁵; n, b and R⁴ are as herein defined. The compounds of formula (II) are useful as intermediates in the preparation of compounds of formula (I).

The present invention is further directed to a process for the preparation of compounds of formula (XVa)

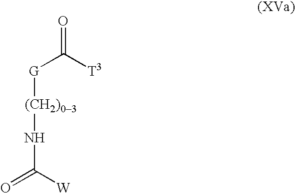

wherein T³₃ G and W are as herein defined. The compounds of formula (XVa) are useful as intermediates in the preparation of compounds of formula (I).

The present invention is further directed to compounds of formula (II)

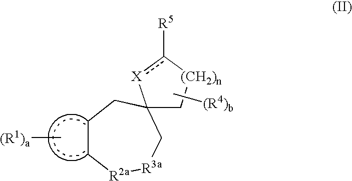

wherein

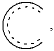

a, R¹, —R^{2a}—R^{3a}, X, [symbol] , R⁵, n, b and R⁴ are as herein defined. The compounds of formula (II) are useful as intermediates in the preparation of compounds of formula (I).

The present invention is further directed to a process for the preparation of (4R)-1,2,3,5-tetrahydro-spiro[4H-1-benzazepine-4,1′-[2]cyclopentene]-3′-carboxylic acid comprising reacting a racemic mixture of 1,2,3,5-tetrahydro-spiro[4H-1-benzazepine-4,1′-[2]cyclopentene]-3′-carboxylic acid with (−)-camphorsulfonic acid. In an embodiment of the present invention, the (−)-camphorsulfonic acid is present in an amount greater than or equal to about one equivalent, preferably about one equivalent.

The present invention is further directed to a process for the preparation of (4S)-1,2,3,5-tetrahydro-spiro[4H-1-benzazepine-4,1′-[2]cyclopentene]-3′-carboxylic acid comprising reacting a racemic mixture of 1,2,3,5-tetrahydro-spiro[4H-1-benzazepine-4,1′-[2]cyclopentene]-3′-carboxylic acid with (+)-camphorsulfonic acid. In an embodiment of the present invention, the (+)-camphorsulfonic acid is present in an amount greater than or equal to about one equivalent, preferably about one equivalent.

The present invention is further directed to novel salts of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, the compound of formula (Ia)

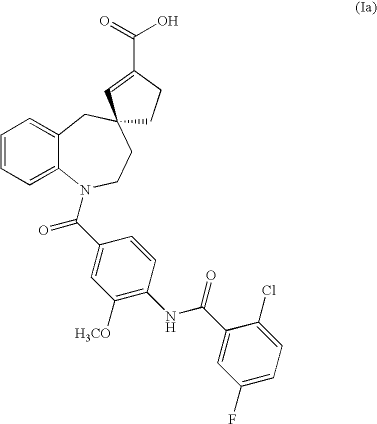

More particularly, the present invention is directed to diethylamine, pipierazine and 1-(2-hydroxyethyl)pyrrolidine salts of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid. Preferably, the diethylamine, piperazine and 1-(2-hydroxyethyl)pyrrolidine salts of the compound of formula (Ia) are crystalline.

The present invention is further directed to a product prepared according to any of the processes described herein.

An embodiment of the present invention is a process for the preparation of a compound of formula (I) wherein

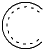

is phenyl, X is —CH₂—, R⁵ is —CO₂H, n is 1, b is 0, —R²—R³— is

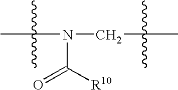

and R¹⁰ is -(3-methoxy-phenyl)-4-(NH—C(O)-(2-chloro-5-fluoro-phenyl)).

Another embodiment of the present invention is a process for the preparation of a compound of formula (II) wherein

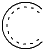

is phenyl, X is —CH₂—, R⁵ is —CO₂H, n is 1, b is 0 and —R^{2a}—R^{3a}— is —NH—CH₂—.

Yet another embodiment of the present invention is a process for the preparation of a compound of formula (XVa) wherein T³ is Cl, G is 1-(3-methoxy-phenyl) and W is 1-(2-chloro-5-fluoro-phenyl).

Yet another embodiment of the present invention is a compound of formula (II) wherein

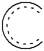

is phenyl, X is —CH₂—, R⁵ is —CO₂H, n is 1, b is 0 and —R^{2a}—R^{3a}— is —NH—CH₂—.

Yet another embodiment of the present invention is a compound of formula (II) selected from the group consisting of a racemic mixture of 1,2,3,5-tetrahydro-spiro[4H-1-benzazepine-4,1′-[2]cyclopentene]-3′-carboxylic acid, (4R)-1,2,3,5-tetrahydro-spiro[4H-1-benzazepine-4,1′-[2]cyclopentene]-3′-carboxylic acid; (4S)-1,2,3,5-tetrahydro-spiro[4H-1-benzazepine-4,1′-[2]cyclopentene]-3′-carboxylic acid and pharmaceutically acceptable salts thereof.

Yet another embodiment of the present invention is 4-(2-chloro-5-fluoro-benzoylamino)-3-methoxy-benzoyl chloride.

The term “halogen” shall include iodine, bromine, chlorine and fluorine.

Unless otherwise noted, “alkyl” and “alkoxy” as used herein, whether used alone or as part of a substituent group, include straight and branched chains having 1 to 8 carbon atoms, as well as cycloalkyl groups containing 3 to 8 ring carbons and preferably 5 to 7 ring carbons, or any number within these ranges. For example, alkyl radicals include methyl, ethyl, n-propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl. Unless otherwise noted, “lower” when used with alkyl shall mean a carbon chain of 1 to 4 carbon atoms. Alkoxy radicals are oxygen ethers formed from the previously described straight, branched, or cyclic chain alkyl groups.

An alkyl as used herein may be substituted with, for example, amino, substituted amino, halogen, hydroxy, heterocyclyl, substituted heterocyclyl, alkyl, alkoxy, alkoxycarbonyl, heteroaryl, substituted heteroaryl, and/or aryl such as phenyl or benzyl.

“Heterocyclyl” or “heterocycle” is a 3- to 8-member saturated or partially saturated single or fused ring system which comprises carbon atoms and from one to three heteroatoms selected from N, O and S. As used herein, “heterocyclyl” or “heterocycle” also refers to 3-, 4-, 7-, or 8-member unsaturated single or fused ring system which comprises carbon atoms and from one to three heteroatoms selected from N, O and S. The heterocyclyl group may be attached at any heteroatom or carbon atom which results in the creation of a stable structure. Examples of heterocyclyl groups include, but are not limited to, pyridine, pyrimidine, oxazoline, pyrrole, imidazole, morpholine, furan, indole, benzofuran, pyrazole, pyrrolidine, piperidine, and benzimidazole.

“Heterocyclyl” or “heterocycle” may be substituted with one or more independent groups including, but not limited to, H, halogen, oxo, OH, alkyl, substituted alkyl, amino, heteroaryl, aldehyde, alkylcarbonyl, alkoxycarbonyl, carboxyl, alkylcarboxyl, alkoxy, and —NZ¹Z² wherein Z¹ and Z² are as described hereinabove.

The term “Ar” or “aryl” as used herein, whether used alone or as part of a substituent group, refers to an aromatic group such as phenyl and naphthyl. Further, “Ph” or “PH” denotes phenyl.

When the Ar or aryl group is substituted, it may have one to three substituents which are independently selected from C₁-C₈ alkyl, C₁-C₈ alkoxy, aralkoxy, substituted C₁-C₈ alkyl (e.g., trifluoromethyl), fluorinated C₁-C₈ alkoxy (e.g., trifluoromethoxy), halogen, cyano, hydroxy, nitro, optionally substituted amino, carboxyl, alkylcarboxyl, alkoxycarbonyl, C₁-C₄ alkylamino (i.e., —NH—C₁-C₄ alkyl), C₁-C₄ dialkylamino (i.e., —N—[C₁-C₄ alkyl]₂ wherein the alkyl groups can be the same or different), —O(CO)O-alkyl, —O-heterocyclyl optionally substituted with optionally substituted alkyl or alkylcarbonyl (for example,

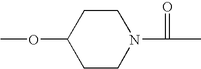

), optionally substituted heteroaryl (for example,

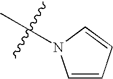

optionally substituted with a group selected from alkyl, substituted alkyl, aldehyde, alkylcarbonyl, carboxyl, alkylcarboxyl, alkoxycarbonyl, and —NZ¹Z² wherein Z¹ and Z² are as described hereinabove), and unsubstituted, mono-, di- or tri-substituted phenyl wherein the substituents on the phenyl are independently selected from aryl, C₁-C₈ alkyl, C₁-C₈ alkoxy, substituted C₁-C₈ alkyl, fluorinated C₁-C₈ alkoxy, halogen, cyano, hydroxy, amino, nitro, carboxyl, alkylcarboxyl, alkylamino, dialkylamino and heteroaryl.

The term “heteroaryl” as used herein represents a stable five or six-membered monocyclic aromatic or nine to ten membered bicyclic aromatic or benzo-fused ring system which comprises carbon atoms and from one to three heteroatoms selected from N, O and S. The heteroaryl group may be attached at any heteroatom or carbon atom which results in the creation of a stable structure. Examples of heteroaryl groups include, but are not limited to pyridinyl, pyrazinyl, pyridazinyl, pyrimidinyl, thiophenyl, furanyl, imidazolyl, isoxazolyl, oxazolyl, pyrazolyl, pyrrolyl, thiazolyl, thiadiazolyl, triazolyl, benzimidazolyl, benzofuranyl, benzothienyl, benzisoxazolyl, benzoxazolyl, benzopyrazolyl, indolyl, benzothiazolyl, benzothiadiazolyl, benzotriazolyl or quinolinyl. Preferred heteroaryl groups include pyridinyl, thiophenyl, furanyl and quinolinyl.

When the heteroaryl group is substituted, the heteroaryl group may have one to three substituents which are independently selected from C₁-C₈ alkyl, substituted C₁-C₈ alkyl, halogen, aldehyde, alkylcarbonyl, aryl, heteroaryl, alkoxy, alkylamino, dialkylamino, arylamino, nitro, carboxyl, alkylcarboxyl, and hydroxy.

The term “aralkoxy” indicates an alkoxy group substituted with an aryl group (e.g., benzyloxy).

The term “Ac” as used herein, whether used alone or as part of a substituent group, means acetyl.

The terms “substituted alkylcarboxy,” “substituted amino,” and “substituted aminocarbonyl” denote substitution of said groups with at least one member selected from halogen, alkyl, substituted alkyl, aryl, alkoxy, amino or substituted amino.

Whenever the term “alkyl” or “aryl” or either of their prefix roots appear in a name of a substituent (e.g., aralkyl, dialkylamino), it shall be interpreted as including those limitations given above for “alkyl” and “aryl.” Designated numbers of carbon atoms (e.g., C₁-C₆) shall refer independently to the number of carbon atoms in an alkyl or cycloalkyl moiety or to the alkyl portion of a larger substituent in which alkyl appears as its prefix root.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “aprotic solvent” shall mean any solvent that does not yield a proton. Suitable examples include, but are not limited to DMF, dioxane, THF, acetonitrile, pyridine, dichloroethane, dichloromethane, MTBE, toluene, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

The term “subject” as used herein, refers to an animal, preferably a mammal, most preferably a human, who is or has been the object of treatment, observation or experiment.

As used herein, “treating” a disorder means eliminating or otherwise ameliorating the cause and/or effects thereof. To “inhibit” or “inhibiting” the onset of a disorder means preventing, delaying or reducing the likelihood of such onset.

As used herein, the term “composition” is intended to encompass a product comprising the specified ingredients in the specified amounts, as well as any product which results, directly or indirectly, from combinations of the specified ingredients in the specified amounts.

Methods are known in the art for determining therapeutically and prophylactically effective doses for the instant pharmaceutical composition.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

The term “prophylactically effective amount” refers to that amount of active compound or pharmaceutical agent that inhibits in a subject the onset of a disorder as being sought by a researcher, veterinarian, medical doctor or other clinician, the delaying of which disorder is mediated by the reduction of increased vascular resistance.

Under standard nomenclature used throughout this disclosure, the terminal portion of the designated side chain is described first, followed by the adjacent functionality toward the point of attachment. Thus, for example, a

“phenylC₁-C₆alkylaminocarbonylC₁-C₆alkyl” substituent refers to a group of the formula

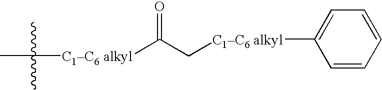

As used herein, in compounds of formula (VIII)

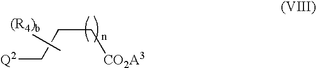

it is intended that the R⁴ groups may be bound at any of the carbon atoms comprising the alkyl portion of the compound of formula (VIII). R⁴ groups may not, therefore be bound to any of the atoms of the Q² of CO₂A³ portions of the compound of formula (VIII). This structural designation of the R⁴ groups shall also extend to compounds of formula (IX) and compounds of formula (X).

Abbreviations used in the specification, particularly the Schemes and Examples, are as follows:

| | |
|---|---|
| Ac = | Acetyl |
| BF₃Et₂O = | Boron Trifluoride Etherate |
| BOC or Boc = | t-Butoxycarbonyl |
| CBz = | Benzyloxycarbonyl |
| CSA = | Camphorsulfonic Acid |
| DBU = | 1,8-Diazabicyclo[5.4.0]undec-7ene |
| DCE = | 1,1-Dichloroethane |
| DCM = | Dichloromethane |
| DIBAL-H = | Diisobutyl aluminum hydride |
| DIPEA or DIEA = | Diisopropylethylamine |
| DMAP = | 4-N,N-Dimethylaminopyridine |
| DMF = | N,N-Dimethylformamide |
| Et = | Ethyl |
| EtOAc = | Ethyl Acetate |
| EtOH = | Ethanol |
| Et₃SiH = | Triethylsilyl Hydride |
| HPLC = | High Pressure Liquid Chromatography |
| KOt-Bu = | Potassium t-butoxide |
| LAH = | Lithium aluminum hydride |
| LDA = | Lithium diisopropylamide |
| Me = | Methyl |
| MeOH = | Methanol |
| MeSO₃H = | Methane sulfonic acid |
| MTBE = | Methyl-t-butyl ester |
| Mtr = | 2,3,6-Trimethyl-4-methoxybenzenesulfonyl |
| Ph = | Phenyl |
| TEA or Et₃N = | Triethylamine |
| TEA = | Trifluoroacetic Acid |
| THF = | Tetrahydrofuran |
| TLC = | Thin Layer Chromatography |
| Ts = | Tosyl (i.e. (4-methylphenyl)sulfonyl) |

The compounds of the present invention may also be present in the form of a pharmaceutically acceptable salt or salts. For use in medicine, the salt or salts of the compounds of this invention refer to non-toxic “pharmaceutically acceptable salt or salts.” Other salts may, however, be useful in the preparation of compounds according to this invention or of their pharmaceutically acceptable salts. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benezenesulfonic, oxalic, pamoic, 2-naphthalenesulfonic, p-toluenesulfonic, cyclohexanesulfamic, salicylic, saccharinic or trifluoroacetic acid. Representative basic/cationic salts include, but are not limited to, benzathine, chloroprocaine, choline, diethanolamine, ethylenediamine, meglumine, procaine, aluminum, calcium, lithium, magnesium, potassium, sodium, or zinc.

Where the compounds according to this invention are chiral, including those that contain at least one stereogenic center, they may accordingly exist as enantiomers. Where stereogenicity extends throughout a plurality of molecular regions, including instances where the compounds possess two or more stereogenic centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the invention give rise to mixture of stereoisomers, these isomers may be separated by conventional techniques such as resolution, for example by formation of diastereomeric salts, kinetic resolution including variants thereof, such as dynamic resolution, preferential crystallization, biotransformation, enzymatic transformation, and preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be separated using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known in the art.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The present invention is directed to a process for preparing compounds of formula (II) as outlined in Scheme 1.

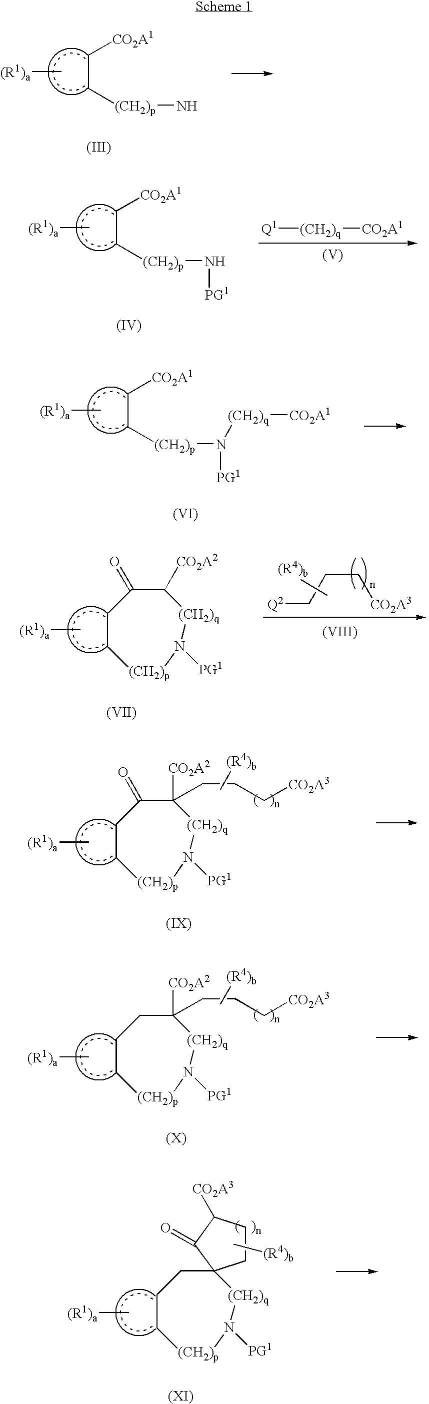

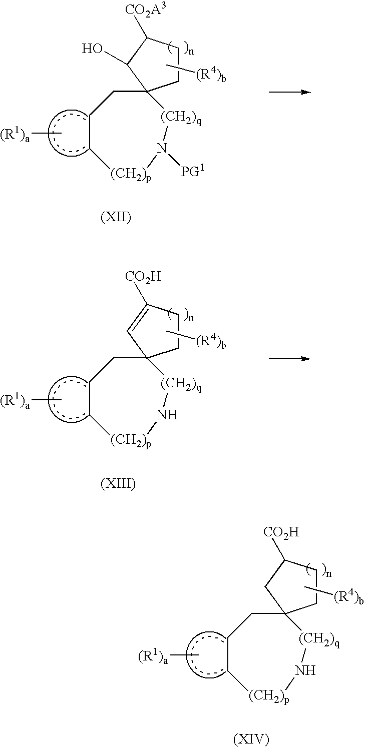

Accordingly, a suitably substituted compound of formula (III), wherein A¹ is a lower alkyl and wherein p is an integer from 0 to 1, a known compound or compound prepared by known methods, is reacted with a suitable amine protecting reagent, under known conditions, to yield the corresponding compound of formula (IV), wherein PG¹ is the corresponding nitrogen protecting group. For example, when the protecting reagent is Boc anhydride, CBz chloride, tosyl chloride or Mtr-chloride, then PG¹ is BOC, CBz, tosyl or Mtr, respectively. Preferably, when p is 0, then PG¹ is selected from tosyl, BOC, CBz or Mtr, more preferably, PG¹ is tosyl. Preferably, when p is 1, then PG¹ is selected from BOC, CBz or Mtr.

The compound of formula (IV) is reacted with a suitably substituted compound of formula (V), wherein A² is a lower alkyl, Q¹ is a leaving group such as a Br, Cl, I, tosylate, mesylate, and the like, and when in the compound of formula (IV) p is 0 then q is 2 and when in the compound of formula (IV) p is 1 then q is 1, a known compound or compound prepared by known methods, in the presence of an inorganic base such as K₂CO₃, Na₂CO₃, Cs₂CO₃, and the like, and mixtures thereof, or a tertiary amine base such as pyridine, TEA, DIPEA, and the like, and mixtures thereof, in an aprotic polar solvent, such as DMF, dioxane, THF, acetonitrile, and the like, and mixtures thereof, to yield the corresponding compound of formula (VI).

The compound of formula (VI) is subjected to ring closure, in the presence of a base such as a sodium or potassium alkoxide (such as sodium methoxide, sodium ethoxide, sodium t-butoxide, potassium methoxide, potassium ethoxide, potassium t-butoxide, and the like, and mixtures thereof), LDA, lithium hexamethyldisilizane, and the like, in an organic solvent such as toluene, THF, t-butanol, and the like, and mixtures thereof, to yield the corresponding compound of formula (VII).

The compound of formula (VII) is reacted with a suitably substituted compound of formula (VIII), wherein A³ is a lower alkyl, Q² is a suitable leaving group such as a Br, Cl, I, tosylate, mesylate, and the like, and wherein n is an integer from 1 to 3, a known compound or compound prepared by known methods, in the presence of a base capable of deprotonating an alpha proton to the ketone on the compound of formula (VII), such as an inorganic base such as K₂CO₃, Na₂CO₃, Cs₂CO₃, and the like, and mixtures thereof, or an organic tertiary amine base such as pyridine, TEA, DIPEA, and the like, and mixtures thereof, or an alkali metal alkoxide such as sodium t-butoxide, potassium t-butoxide, sodium methoxide, and the like, and mixtures thereof, in an aprotic solvent such as DMF, dioxane, THF, acetonitrile, and the like, and mixtures thereof, to yield the corresponding compound of formula (IX).

The compound of formula (IX) is reduced with a suitable reducing agent such as trimethylsilane, triethylsilane, LAH, borane THF complex, and the like, in the presence of a Lewis acid such as BF₃.Etherate, titanium tetrachloride, and the like, optionally in the presence of an acid such as TFA, methanesulfonic acid, trifluoromethanesulfonic acid (triflic acid), and the like, and mixtures thereof, in a halogenated organic solvent such as dichloroethane, dichloromethane, and the like, and mixtures thereof, to yield the corresponding compound of formula (X). In one embodiment, the compound of formula (IX) was reduced by reacting with triethylsilane in the presence of BF₃.Etherate, TFA and methanesulfonic acid. For example, the compound of formula (IX) was reduced by reacting with triethylsilane in the presence of BF₃.Etherate, TFA and methanesulfonic acid, wherein the triethylsilane, BF₃.Etherate, TFA and methanesulfonic acid were present in a molar ratio of 3.75 to 2.79 to 5.27 to 1.2, respectively. In another example, the triethylsilane, BF₃.Etherate, TFA and methanesulfonic acid were present in a molar ratio of 5.0 to 1.8 to 2.5 to 6.0, respectively.

Alternatively, the compound of formula (XI) is reduced by reacting with hydrogen gas, in the presence of a catalyst such as Pd on carbon, PtO₂, Raney Nickel, and the like, in the presence of a Brönsted acid such as acetic acid, sulfuric acid, and the like, in an alcoholic organic solvent such as methanol, ethanol, and the like, and mixtures thereof, to yield the corresponding compound of formula (X).

Preferably, the reducing agent preferentially reduces and deoxygenates the —C(O)— to a —CH₂— over reducing the —CO₂A² and/or CO₂A³ ester group.

The compound of formula (X) is subjected to ring closure, in the presence of a base capable of deprotonating an alpha proton to the CO₂A³ substituent such as an alkali metal alkoxide (such as a sodium or potassium alkoxide such as sodium methoxide, sodium ethoxide, sodium t-butoxide, potassium methoxide, potassium ethoxide, potassium t-butoxide, and the like, and mixtures thereof), LDA, lithium hexamethyldisilizane, and the like, in an organic solvent that does not prevent the deprotonation of an alpha proton to the CO₂A³ substituent such as toluene, THF, t-butanol, and the like, and mixtures thereof, preferably in an aprotic organic solvent such as THF, toluene, and the like, and mixtures thereof, to yield the corresponding compound of formula (XI).

The compound of formula (XI) is reduced with a suitable reducing agent such as sodium borohydride, diisobutyl aluminum hydride (DiBAL-H), and the like, in a organic solvent such as ethanol, methanol, THF, and the like, and mixtures thereof, to yield the corresponding compound of formula (XII).

Alternatively, the compound of formula (XI) is reduced by reacting with hydrogen gas, in the presence of a catalyst such as Raney Nickel, and the like, in an alcoholic organic solvent such as methanol, ethanol, and the like, and mixtures thereof, to yield the corresponding compound of formula (XII).

Preferably, the reducing agent preferentially reduces and deoxygenates the —C(O)— to —CH(OH)— over reducing the CO₂A³ ester group.

The compound of formula (XII) is reacted according to known methods, to yield the corresponding compound of formula (XIII). More particularly, the compound of formula (XII) is converted to the compound of formula (XIII) using one or more steps to (a) hydrolyze the —CO₂A³ ester to the corresponding —CO₂H, for example by reacting with water, catalyzed by a strong acid such as H₂SO₄, HCl, and the like or mixtures thereof; or by reacting with water, catalyzed by a strong base such as NaOH, LiOH, KOH, and the like, and mixtures thereof, (b) dehydrate to form a conjugated double bond, for example by reacting with a strong acid such as H₂SO₄, HCl, and the like, and mixtures thereof; or by reacting with mesyl chloride in the presence of an organic base such as DBU, DMAP, TEA, pyridine, and the like, and mixtures thereof, and (c) de-protect the —N-PG¹ group to the corresponding —NH, for example by reacting with a strong acid such as H₂SO₄, HCl, and the like, and mixtures thereof; or when PG¹ is CBz, by hydrogenation with hydrogen gas, in the presence of a suitable catalyst such as Pd on carbon. One skilled in the art will recognize that the above steps may be performed in any order which will yield the corresponding compound of formula (XIII).

Preferably, the protecting group PG¹ is a protecting group which may be removed under acidic conditions and the compound of formula (XII) is reacted to yield the compound of formula (XIII) in one step, by reacting the compound for formula (XII) with a strong acid (i.e an acid capable of carrying out the dehydration to a conjugated double bond, de-protection of the nitrogen and the hydrolysis of the ester to the carboxylic acid in the compound of formula (XII)), such as sulfuric acid, hydrochloric acid, and the like, and mixtures thereof, in a polar organic solvent such as acetic acid, and the like, preferably at an elevated temperature in the range of from about room temperature to about 140° C., more preferably at about 100° C., to yield the corresponding compound of formula (XIII). Alternatively, the compound of formula (XII) is reacted with Eaton's acid to yield the compound of formula (XIII).

The compound of formula (XIII) is further optionally reduced using hydrogen gas or a suitable source of hydrogen such as triethylsilane, dimethylphenylsilane, HCOONH₄, in the presence of a suitable catalyst such as Pd on carbon, Raney nickel, Rh(P(C₆H₅)₃)₃, PtO₂, RhCl(P(C₆H₅)₃)₃, and the like, and mixtures thereof, in an organic solvent such as ethyl acetate, THF, methanol, ethanol, and the like, and mixtures thereof, to yield the corresponding compound of formula (XIV).

Alternatively, the compound of formula (XIII) may be optionally reacted with sodium borohydride, in an organic solvent such as methanol, THF, and the like, and mixtures thereof, to yield the corresponding compound of formula (XIV).

One skilled in the art will recognize that compounds of formula (XI) may be de-protected and optionally hydrolyzed by known methods (for example as described above), to yield the corresponding compound of formula (II) wherein R⁵ is alkoxycarbonyl or carboxylic acid and X is C(O). Compounds of formula (XII) may be de-protected and optionally hydrolyzed (for example as described above), to yield the corresponding compound of formula (II) wherein R⁵ is alkoxycarbonyl or carboxylic acid and X is CHOH.

One skilled in the art will further recognize that compounds of formula (XIII) correspond to compounds of formula (II) wherein R⁵ is carboxyl or alkoxycarbonyl and X is CH. Similarly, compounds of formula (XIV) correspond to compounds of formula (II) wherein R⁵ is carboxyl or alkoxycarbonyl and X is CH₂.

One skilled in the art will further recognize that compounds of formula (II) wherein R⁵ is other than alkoxycarbonyl or carboxyl may be prepared from the corresponding compound of formula (II) wherein R⁵ is carboxyl by known methods.

Preferably, the compound of formula (II) is resolved into its corresponding enantiomers (when [symbol] is a double bond) or diastereomers (when [symbol] is a single bond) by known methods, for example by column chromatography, selective recrystallization or by resolution with a suitable resolving agent such as (−)-camphorsulfonic acid, (+)-camphosulfonic acid, D-tartaric acid or L-tartaric acid, and the like. One skilled in the art will recognize that when the compound of formula (II) is a mixture of enantiomers, the enantiomers may be separated using classical resolution or by selective recrystallization by first converted the enantiomers into diastereomers using a chiral auxiliary followed by selective recrystallization or column chromatographic separation of the diastereomers and re-generation of the original enantiomers.

The present invention is further directed to a process for preparing compounds of formula (I) as outlined in Scheme 2.

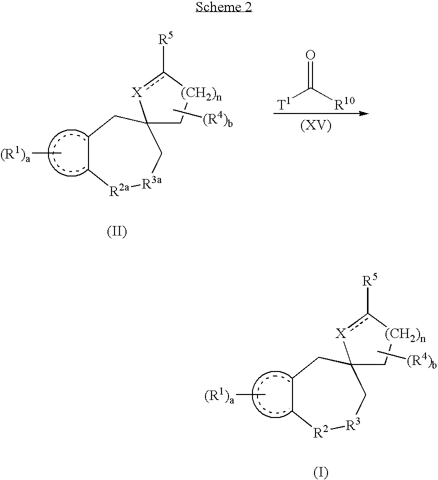

Accordingly, a suitably substituted compound of formula (II), a compound prepared as in Scheme 1 above, is reacted with a suitable substituted compound of formula (XV), wherein T¹ is Cl, Br or F, preferably, T¹ is Cl, a known compound or compound prepared by known methods, in the presence of a base capable of neutralizing HT¹; preferably, the base capable of neutralizing HT¹ does not react with the compound of formula (XV), such as an organic tertiary amine base such as TEA, DIPEA, pyridine, and the like or an inorganic base such as K₂CO₃, Na₂CO₃, NaHCO₃, NaOH, KOH, and the like, in a non-alcoholic organic solvent such as THF, dichloroethane, dichloromethane, toluene, pyridine, and the like or a mixture of a non-alcoholic organic solvent and water such as a THF/water mixture, and the like, wherein said mixture may be biphasic, preferably at a temperature in the range of between about 0° C. and about room temperature, to yield the corresponding compound of formula (I).

One skilled in the art will recognize that when in the compound of formula (II) R⁵ is carboxyl or other reactive group, said carboxyl or reactive group is preferably protected prior to the reaction with the compound of formula (XV) and the protecting group removed after reacting with the compound of formula (XV), to yield the corresponding compound of formula (I). For example, wherein the R⁵ group is carboxyl, the carboxyl may be protected by reacting the corresponding compound of formula (II) with TMSCl in situ; or the carboxyl may be protected as a lower alkyl ester.

One skilled in the art will further recognize that wherein the compound of formula (I) R⁵ is carboxyl or alkoxycarbonyl, the compound of formula (I) may be further optionally reacted according to known methods to transform the R⁵ carboxyl or alkoxycarbonyl group to alkyl, substituted alkyl, aldehyde, substituted alkoxycarbonyl, —(CH₂)ₖNZ¹Z² or —C(O)NZ¹Z².

Alternatively, compounds of formula (I) may be prepared by reacting the compound of formula (III) with a suitably substituted acid halide, a compound of the formula (XV)

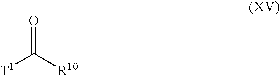

in the presence of a base such as TEA, DPEA, pyridine, and the like, and mixtures thereof, in an aprotic organic solvent such as THF, dichloroethane, dichloromethane, toluene, pyridine, and the like, and mixtures thereof, to yield the corresponding compound of formula (IV) wherein the —C(O)—R¹⁰ substituent is the PG¹ group, a compound of the formula (IVa)

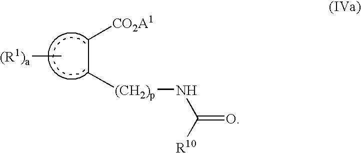

The compound of formula (IVa) may then be reacted according to the process outlined in Scheme 1 to yield the corresponding compounds of formulae (VI), (VII), (IX), (X), (XI) and (XII) wherein the —C(O)—R¹⁰ substituent is the PG¹ group.

Compounds of formula (XV) wherein R¹⁰ is -G-E-W and wherein G is as previously defined, E is —NH—C(O)— and W is selected from the group consisting of alkyl, substituted alkyl, amino, substituted amino, alkylthiophenyl, alkyl-sulfoxidephenyl, aryl, substituted aryl, heteroaryl or substituted heteroaryl; may be prepared according to the process outlined in Scheme 3.

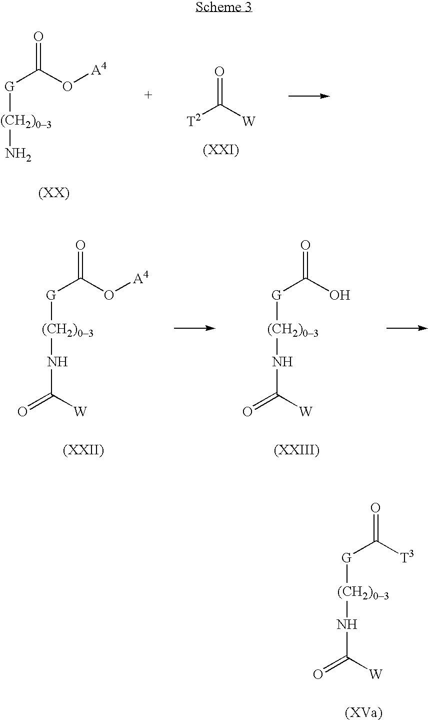

Accordingly, a suitably substituted compound of formula (XX), wherein A⁴ is lower alkyl, a known compound or compound prepared by known methods, is reacted with a suitably substituted compound of formula (XXI) wherein T² is Cl, Br or F, preferably T² is Cl, a known compound or compound prepared by known methods, in the presence of a base capable of neutralizing HT²; preferably the base capable of neutralizing HT² does not react with the compound of formula (XV), such as an organic tertiary amine base such as TEA, DIPEA, pyridine, and the like, and mixtures thereof, or an inorganic base such as K₂CO₃, Na₂CO₃, NaHCO₃, NaOH, KOH, and the like, and mixtures thereof, in a non-alcoholic organic solvent such as ethyl acetate, THF, methylenechloride, dichloroethane, dichloromethane, toluene, benzene, pyridine, and the like, and mixtures thereof, or a mixture of a non-alcoholic organic solvent and water such as a THF/water mixture, and the like, wherein said mixture may be biphasic, preferably at a temperature in the range of between about 0° C. and about room temperature, to yield the corresponding compound of formula (XXII).

The compound of formula (XXII) is hydrolyzed by reacting with water in the presence of a base such as NaOH, KOH, LiOH, and the like, and mixtures thereof, in an organic solvent such as methanol, ethanol, THF, dioxane, and the like, and mixtures thereof, to yield the corresponding compound of formula (XXIII).

The compound of formula (XXIII) is reacted with a reagent capable of converting the —CO₂H (carboxyl group) to the corresponding —C(O)T³ (i.e. an acid halide group such as —C(O)Cl, —C(O)Br or —C(O)F), such as oxalyl chloride, thionyl chloride, thionyl bromide, phosphorous tribromide, SF₄, cyanuric fluoride, and the like, preferably oxalyl chloride, in an inert organic solvent such as DCM, DCE, toluene, and the like, preferably at a temperature in the range of between about 0° C. and about room temperature, to yield the corresponding compound of formula (XVa), wherein T³ is the corresponding halide anion. For example when the compound of formula (XXIII) is reacted with oxalyl chloride, T³ is Cl.

One skilled in the art will recognize that wherein a reaction step of the present invention may be carried out in a variety of solvents or solvent systems, said reaction step may also be carried out in a mixture of the suitable solvents or solvent systems.

The present invention is further directed to novel salts of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid (the compound of formula (Ia)). More specifically, the present invention is directed to diethylamine, pipierazine and 1-(2-hydroxyethyl)pyrrolidine salts of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid.

Powder X-ray diffraction patterns for the salts of the present invention were measured as follows. The salt sample was backloaded into a conventional X-ray holder and analyzed as received. Using an X-Celerator detector, the sample was scanned from 3 to 35 °2θ at a step size of 0.0165 °2θ and a time per step of 10.16 seconds. The effective scan speed was 0.2067°/s. Instrument voltage and current settings of 45 kV and 40 mA were employed.

In an embodiment of the present invention is a diethylamine salt of the compound of formula (Ia), wherein the molar ratio of the compound of formula (Ia) to diethylamine is 1:1. Preferably, the diethylamine salt of the compound of formula (Ia) is crystalline.

The diethylamine salt of the compound of formula (Ia) may be prepared by reacting the compound of formula (Ia) with diethylamine; in a mixture of (a) a polar solvent or mixture thereof, such as methanol, ethanol, and the like, and (b) an anti-solvent or mixture thereof, such as ethanol, heptane, ethyl acetate, isopropylacetate, t-butyl-methylether (MTBE), and the like, for example in a mixture of methanol/ethanol, methanol/isopropylacetate, methanol/MTBE, and the like; and then separating the salt, such as by precipitating the solid, preferably by cooling or evaporating the solvents, at least partially.

An embodiment of the crystalline diethylamine salt of the compound of formula (Ia) may be characterized by its X-ray diffraction pattern, as listed in Table A1, below.

TABLE A1
X-Ray Diffraction Pattern. Diethylamine Salt

| Position [°2θ] | d-spacing [Å] | Relative Intensity [%] |
|---|---|---|
| 12.4469 | 7.1116 | 13.10 |
| 12.6297 | 7.0091 | 5.29 |
| 12.9474 | 6.8377 | 2.76 |
| 13.2274 | 6.6936 | 2.06 |
| 13.6758 | 6.4751 | 15.99 |
| 13.9948 | 6.3283 | 45.16 |
| 14.8109 | 5.9814 | 5.66 |
| 14.928 | 5.9348 | 3.38 |
| 15.3664 | 5.7664 | 1.98 |
| 15.5378 | 5.7031 | 5.27 |
| 16.0254 | 5.5307 | 29.23 |
| 16.4868 | 5.3769 | 15.27 |
| 17.1962 | 5.1567 | 60.20 |
| 17.6157 | 5.0348 | 18.08 |
| 18.0770 | 4.9074 | 4.52 |
| 18.4150 | 4.8181 | 3.81 |
| 18.7511 | 4.7324 | 1.78 |
| 19.0004 | 4.6709 | 9.96 |
| 19.2580 | 4.6090 | 10.28 |
| 19.6077 | 4.5276 | 2.48 |
| 20.2682 | 4.3815 | 78.24 |
| 20.7710 | 4.2766 | 19.85 |
| 21.1852 | 4.1939 | 50.33 |
| 21.9320 | 4.0527 | 2.27 |
| 22.4210 | 3.9654 | 14.46 |
| 23.1866 | 3.8330 | 44.46 |
| 23.2845 | 3.8203 | 50.44 |
| 23.7616 | 3.7447 | 44.86 |
| 24.1721 | 3.6820 | 38.54 |
| 24.5539 | 3.6256 | 13.46 |
| 25.4790 | 3.4960 | 20.14 |
| 26.4543 | 3.3693 | 100.00 |
| 27.2074 | 3.2777 | 47.26 |
| 27.6733 | 3.2236 | 24.27 |
| 28.3885 | 3.1440 | 7.76 |
| 28.8110 | 3.0988 | 6.05 |
| 29.3616 | 3.0420 | 15.82 |
| 30.2239 | 2.9571 | 7.43 |
| 30.8063 | 2.9001 | 2.11 |
| 31.1702 | 2.8671 | 2.75 |
| 31.2943 | 2.8631 | 3.18 |
| 31.9613 | 2.7979 | 10.81 |
| 32.3129 | 2.7683 | 11.11 |
| 33.0705 | 2.7066 | 5.45 |
| 33.3536 | 2.6842 | 4.25 |
| 34.0349 | 2.6320 | 2.86 |
| 34.5735 | 2.5923 | 2.99 |

An embodiment of the present invention is a crystalline diethylamine salt of the compound of formula (Ia) characterized by the major X-ray diffraction peaks having a relative intensity of greater than or equal to about 10%, as listed in Table A1, above.

Another embodiment of the present invention is a piperazine salt of the compound of formula (Ia), wherein the molar ratio of the compound of formula (Ia) to piperazine is 2:1. Preferably, the piperazine salt of the compound of formula (Ia) is crystalline.

The piperazine salt of the compound of formula (Ia) may be prepared by reacting the compound of formula (Ia) with piperazine; in a mixture of (a) a polar solvent or mixture thereof, such as methanol, ethanol, and the like, and (b) an anti-solvent or mixture thereof, such as ethanol, heptane, ethyl acetate, isopropylacetate, t-butyl-methylether (MTBE), and the like, for example in a mixture of methanol/ethanol, methanol/isopropylacetate, methanol/MTBE, and the like; and then separating the salt, such as by precipitating the solid, preferably by cooling or evaporating the solvents, at least partially.

An embodiment of the crystalline piperazine salt of the compound of formula (Ia) may be characterized by its X-ray diffraction pattern, as listed in Table A2, below.

TABLE A2
X-Ray Diffraction Pattern, Piperazine Salt

| Position [°2θ] | d-spacing [Å] | Relative Intensity [%] |
|---|---|---|
| 13.5395 | 6.5400 | 21.78 |
| 14.1884 | 6.2423 | 8.96 |
| 14.8734 | 5.9564 | 25.04 |
| 15.2444 | 5.8122 | 4.23 |
| 15.4039 | 5.7524 | 12.84 |
| 15.8609 | 5.5877 | 52.16 |
| 16.5948 | 5.3421 | 15.00 |
| 16.8391 | 5.2652 | 4.36 |
| 16.9688 | 5.2253 | 2.34 |
| 17.1351 | 5.1749 | 2.61 |
| 17.5825 | 5.0442 | 4.27 |
| 17.6821 | 5.0161 | 9.08 |
| 17.9503 | 4.9417 | 4.76 |
| 18.5405 | 4.7857 | 31.72 |
| 19.1470 | 4.6355 | 92.10 |
| 19.6968 | 4.5073 | 55.49 |
| 20.1348 | 4.4102 | 68.92 |
| 20.7233 | 4.2863 | 12.82 |
| 21.3009 | 4.1714 | 10.41 |
| 22.1553 | 4.0124 | 17.97 |
| 22.8971 | 3.8841 | 29.11 |
| 23.9903 | 3.7095 | 24.43 |
| 24.7962 | 3.5907 | 27.08 |
| 25.8556 | 3.4460 | 100.00 |
| 26.5390 | 3.3587 | 40.92 |
| 27.1754 | 3.2815 | 41.38 |
| 27.5201 | 3.2412 | 40.85 |
| 28.9219 | 3.0872 | 26.04 |
| 30.0687 | 2.9720 | 14.28 |
| 30.7142 | 2.9110 | 6.56 |
| 31.6571 | 2.8264 | 10.40 |
| 26.4543 | 3.3693 | 100.00 |
| 33.5897 | 2.6681 | 10.85 |
| 34.2770 | 2.6162 | 6.13 |

An embodiment of the present invention is a crystalline piperazine salt of the compound of formula (Ia) characterized by the major X-ray diffraction peaks having a relative intensity of greater than or equal to about 10%, as listed in Table A2, above.

Another embodiment of the present invention is a 1-(2-hydroxyethyl)pyrrolidine salt of the compound of formula (Ia), wherein the molar ratio of the compound of formula (Ia) to 1-(2-hydroxyethyl)pyrrolidine is 1:1. Preferably, the 1-(2-hydroxyethyl)pyrrolidine salt of the compound of formula (Ia) is crystalline.

The 1-(2-hydroxyethyl)pyrrolidine salt of the compound of formula (Ia) may be prepared by reacting the compound of formula (Ia) with 1-(2-hydroxyethyl)pyrrolidine; in a mixture of (a) a polar solvent or mixture thereof, such as methanol, ethanol, and the like, and (b) an anti-solvent or mixture thereof, such as ethanol, heptane, ethyl acetate, isopropylacetate, t-butyl-methylether (MTBE), and the like, for example in a mixture of methanol/ethanol, methanol/isopropylacetate, methanol/MTBE, and the like; and then separating the salt, such as by precipitating the solid, preferably by cooling or evaporating the solvents, at least partially.

An embodiment of the crystalline 1-(2-hydroxyethyl)pyrrolidine salt of the compound of formula (Ia) may be characterized by its X-ray diffraction pattern, as listed in Table A3, below.

TABLE A3
X-Ray Diffraction Pattern, 1-(2-hydroxyethyl)pyrrolidine Salt

| Position [°2θ] | d-spacing [Å] | Relative Intensity [%] |
|---|---|---|
| 12.4052 | 7.1353 | 35.63 |
| 12.8369 | 6.8964 | 5.59 |
| 12.9433 | 6.8399 | 7.08 |
| 13.2908 | 6.6619 | 9.15 |
| 14.5331 | 6.0950 | 27.39 |
| 15.8254 | 5.6001 | 100.00 |
| 16.1407 | 5.4914 | 25.15 |
| 17.0466 | 5.2016 | 10.01 |
| 17.5261 | 5.0604 | 36.71 |
| 18.0214 | 4.9224 | 9.08 |
| 18.5465 | 4.7842 | 6.78 |
| 18.8205 | 4.7151 | 33.63 |
| 19.3437 | 4.5888 | 10.85 |
| 19.6767 | 4.5119 | 16.22 |
| 20.0173 | 4.4358 | 17.78 |
| 20.4608 | 4.3407 | 29.62 |
| 20.6769 | 4.2958 | 23.59 |
| 21.7248 | 4.0909 | 16.51 |
| 22.1398 | 4.0152 | 21.99 |
| 22.6780 | 3.9211 | 86.85 |
| 23.3486 | 3.8100 | 56.43 |
| 23.9247 | 3.7195 | 75.49 |
| 24.4967 | 3.6339 | 36.16 |
| 25.0891 | 3.5495 | 24.11 |
| 25.3622 | 3.5119 | 36.04 |
| 25.9050 | 3.4395 | 6.56 |
| 26.5362 | 3.3591 | 6.89 |
| 26.8162 | 3.3247 | 6.40 |
| 27.6456 | 3.2268 | 17.69 |
| 28.3619 | 3.1469 | 8.49 |
| 29.1634 | 3.0622 | 31.69 |
| 30.3545 | 2.9447 | 7.17 |
| 31.2668 | 2.8608 | 7.26 |
| 32.5468 | 2.7512 | 13.63 |
| 33.3510 | 2.6867 | 10.68 |
| 33.9666 | 2.6394 | 5.56 |

An embodiment of the present invention is a crystalline 1-(2-hydroxyethyl)pyrrolidine salt of the compound of formula (Ia) characterized by the major X-ray diffraction peaks having a relative intensity of greater than or equal to about 10%, as listed in Table A3, above.

The following Examples are set forth to aid in the understanding of the invention, and are not intended and should not be construed to limit in any way the invention set forth in the claims which follow thereafter.

### EXAMPLE 1

### Ethyl 4-(4-ethoxy-4-oxobutyl)-2,3,4,5-tetrahydro-1-[(4-methylphenyl)sulfonyl]-5-oxo-1H-1-benzazepine-4-carboxylate

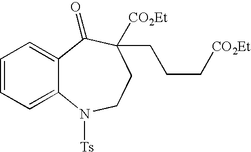

To a suspension of ethyl 2,3,4,5-tetrahydro-1-[(4-methylphenyl)sulfonyl]-5-oxo-1H-1-benzazepine-4-carboxylate (3.41 mol, 1323 g) and ethyl 4-bromobutyrate (3.41 mol, 667 g) in DMF (4787 g) at the room temperature was added K₂CO₃ (6.87 mol, 950 g) in one portion. The resulting suspension was stirred at the room temperature for 24 hours. HPLC analysis of the reaction mixture showed that the reaction was completed. The reaction solution was diluted with EtOAc (11391 g), quenched with 32-34% of aqueous HCl solution (1680 g), and further diluted with H₂O (9259 g). After separation of the layers, the organic layer was washed with H₂O (9259 g), saturated NaHCO₃ solution (5054 g), brine (5054 g), and dried over Na₂SO₄ (1686 g). After filtration and concentration, the yellow oil was crystallized from EtOH (6178 g). The product was isolated as a white solid.

mp 86-88.5° C.

¹H NMR (CDCl₃, 300 MHz) δ7.55 (d, J=8.3 Hz, 2H), 7.35 (m, 4H), 7.26 (d, J=8.3 Hz, 2H), 4.11 (m, 4H), 3.96 (m, 2H), 2.44 (m, 1H), 2.42 (s, 3H), 2.60 (td, J=7.0, 1.4 Hz, 2H), 1.87 (m, 2H), 1.64 (m, 1H), 1.49 (m, 2H), 1.25 (t, J=7.1 Hz, 3H), 1.09 (t, J=7.1 Hz, 3H)

MS (ESI): m/z 501.9 (MH⁺)

Elemental analysis for C₂₆H₃₁NO₇S: Calc'd: C, 62.26; H, 6.23; N, 2.79; S, 6.39. Found: C, 62.15; H, 6.27; N, 2.75; S, 6.44.

### EXAMPLE 2

### Ethyl 4-(4-ethoxy-4-oxobutyl)-2,3,4,5-tetrahydro-1-[(4-methylphenyl)sulfonyl]-1H-1-benzazepine-4-carboxylate

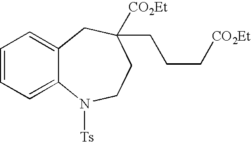

To a 12 L, 3-necked round-bottomed flask equipped with a mechanical stirrer, a condenser, a thermocouple, and an N₂-inlet was added ethyl 4-(4-ethoxy-4-oxobutyl)-2,3,4,5-tetrahydro-1-[(4-methyl phenyl)sulfonyl]-5-oxo-1H-1-benzazepine-4-carboxylate (0.60 mol, 300.15 g) and 1.5 liters of dichloroethane. The solution was cooled in an ice-water bath to 3-5° C. To the stirred solution was added Et₃SiH (2.25 mol, 360 mL) in one portion, followed by the addition of TFA (1.11 mol, 85.9 mL), BF₃.Et₂O (0.72 mol, 91.2 mL), and MeSO₃H (3.16 mol, 2.5.5 mL). The resulting pale yellow solution was stirred and the internal reaction temperature rose from 5° C. to 30° C. in 10 minutes. The reaction temperature then slowly decreased to 6-8° C. after 30 minutes of stirring. The ice-water bath was removed and the reaction solution was stirred at the room temperature for 2 hours. TLC analysis of the reaction mixture indicated that the reaction was completed. The reaction mixture was diluted with H₂O (750 mL) and stirred for 15 minutes. The aqueous layer was separated and the organic layer was washed with H₂O (600 mL), a saturated solution of NaHCO₃ (600 mL), and brine (1000 mL). The organic solution was dried with MgSO₄. After filtration and concentration, the solvent was removed in vacuo to yield the crude product (276.4 g) as colorless oil. The crude product was used for the next step without any further purification.

¹H NMR (CDCl₃, 300 MHz) δ7.61 (d, J=8.3 Hz, 2H), 7.25 (m, 3H), 7.12 (m, 3H), 4.10 (q, J=7.1 Hz, 2H), 3.97 (br m, 3H), 3.53 (br m, 1H), 2.52 (m, 2H), 2.42 (s, 3H), 2.21 (br m, 3H), 1.70 (br m, 1H), 1.50 (br m, 2H), 1.43 (br m, 2H), 1.24 (t, J=7.1 Hz, 3H), 1.12 (t, J=7.1 Hz, 3H)

MS (ESI): m/z 488.4 (MH⁺)

Elemental analysis for C₂₆H₃₃NO₆S: Calc'd: C, 64.04; H, 6.82; N, 2.87; S, 6.58. Found: C, 64.28; H, 6.76; N, 2.68; S, 6.46.

### EXAMPLE 3

### Ethyl 1,2,3,5-tetrahydro-1-[(4-methyl phenyl)sulfonyl]-2′-oxo-spiro[4H-1-benzazepine-4,1′-cyclopentane]-3′-carboxylate

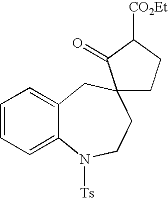

To a 5 L, 3-necked round-bottomed flask equipped with a mechanical stirrer, a condenser, a thermocouple, and an N₂-inlet was added a solution of ethyl 4-(4-ethoxy-4-oxobutyl)-2,3,4,5-tetrahydro-1-[(4-methylphenyl)sulfonyl]-1H-1-benzazepine-4-carboxylate (1.03 mol, 505.3 g) in toluene (2.5 L). To the stirred solution was added KOt-Bu (1.55 mol, 174.4 g) in two equal portions 30 minutes apart. The temperature of the reaction rose from the room temperature to 36° C. in about 20 minutes and the reaction solution became amber color from a colorless solution. The reaction solution was stirred at the room temperature for 20 hours. The reaction mixture was cooled in an ice-water bath to 10-15° C., acidified with 2N HCl solution (1 L), and stirred for 15 minutes. After separation of layers, the organic layer was washed twice of H₂O (1 L), brine (1 L), and dried over MgSO₄. After filtration and concentration, the solvent was removed in vacuo at 60° C. The crude product was obtained as pale yellow oil. The crude product was used for the next step without any further purification.

¹H NMR (CDCl₃, 300 MHz) δ7.58 (d, J=8.2 Hz, 2H), 7.40 (d, J=7.5 Hz, 1H), 7.24 (m, 4H), 7.01 (m, 1H), 4.22 (m, 1H), 4.17 (q, J=7.1 Hz, 2H), 3.27 (m, 2H), 2.41 (s, 3H), 2.34 (m, 1H), 2.13 (m, 4H), 1.63 (m, 0.1H), 1.52 (m, 2H), 1.27 (t, J=7.1 Hz, 3H)

MS (ESI): m/z 442.0 (MH⁺)

Elemental analysis for C₂₄H₂₇NO₅S: Calc'd: C, 65.28; H, 6.16; N, 3.17; S, 7.26. Found: C, 65.25; H, 6.29; N, 3.20; S, 7.37.

In a repeat of the above experiment, the crude was further crystallized from methanol to yield a white solid (mp 114-116° C.).

### EXAMPLE 4

### Ethyl 1,2,3,5-tetrahydro-2′-hydroxy-1-[(4-methylphenyl)sulfonyl]-spiro[4H-1-benzazepine-4,1′-cyclopentane]-3′-carboxylate

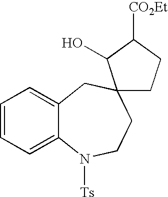

To a 12 L, 3-necked round-bottomed flask equipped with a mechanical stirrer, a condenser, a thermocouple, and an N₂-inlet was added a solution of ethyl 1,2,3,5-tetrahydro-1-[(4-methyl phenyl)sulfonyl]-2′-oxo-spiro[4H-1-benzazepine-4,1′-cyclopentane]-3′-carboxylate (0.92 mol, 404.7 g) in methanol (6 L). The solution was cooled to 5-7° C. in an ice-water bath while stirring and NaBH₄ (0.63 mol, 23.95 g) was added in two equal portions 30 minutes apart. The temperature of the reaction solution was rose to 12-14° C. after each addition. The reaction solution was stirred in an ice-water bath for 30 minutes after addition. TLC analysis of the reaction solution indicated the reaction was completed. 1.5N HCl solution (1.5 L) was slowly added over 15 minutes to quench the reaction. The quenched solution was stirred in an ice-water bath for 15 minutes and at the room temperature for 1 hour. 80% of methanol was removed in vacuo. The residue was diluted with EtOAc (4 L) and H₂O (2 L). After stirred for 15 minutes, the layers were separated. The aqueous layer was extracted with EtOAc (2 L). The combined organic layer was washed with H₂O (2 L), a saturated NaHCO₃ solution (2 L), H₂O (2 L), brine (2 L), and dried with MgSO₄. After filtration and concentration, the crude product was obtained as white semi-solid. The crude product was used for the next step without any further purification. The product could be crystallized from EtOAc/hexane (1/3) to give a white solid (mp 139-140° C.) if necessary.

¹H NMR (CDCl₃, 300 MHz) δ7.56 (d, J=8.2 Hz, 2H), 7.35 (m, 1H), 7.22 (m, 3H), 7.17 (m, 2H), 4.15 (q, J=7.1 Hz, 2H), 3.87 (br m, 1H), 3.71 (m, 1H), 3.57 (br m, 1H), 3.02 (br s, 1H), 2.91 (m, 1H), 2.43 (br s, 1H), 2.41 (s, 3H), 2.27 (m, 1H), 2.05 (m, 1H), 1.88 (m, 1H), 1.57 (m, 2H), 1.45 (m, 1H), 1.26 (m, 1H), 1.24 (t, J=7.1 Hz, 3H)

MS (ESI): m/z 443.9 (MH⁺)

Elemental analysis for C₂₄H₂₉NO₅S: Calc'd: C, 64.99; H, 6.59; N, 3.16; S, 7.23. Found: C, 64.96; H, 6.69; N, 3.06; S, 7.06.

In a repeat of the above experiment, the crude was further crystallized from EtOAc/hexane (1/3) to give a white solid (mp 139-140° C.).

### EXAMPLE 5

### 1,2,3,5-tetrahydro-spiro[4H-1-benzazepine-4,1′-[2]cyclopentene]-3′-carboxylic acid

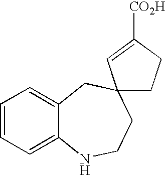

To a 12 L, 3-necked round-bottomed flask equipped with a mechanical stirrer, a condenser, a thermocouple, and an N₂-inlet was added a solution of ethyl 1,2,3,5-tetrahydro-2′-hydroxy-1-[(4-methyl phenyl)sulfonyl]-spiro[4H-1-benzazepine-4,1′-cyclopentane]-3′-carboxylate (1.1 mol, 487.9 g) in acetic acid (950 mL). To the solution was added concentrated H₂SO₄ (475 mL) in one portion. The pale yellow solution became dark brown and the temperature of the reaction mixture rose to 80° C. The reaction was warmed to 100° C. and stirred for 22-24 hours. The dark brown solution was cooled to the room temperature and diluted with cold H₂O (1 L). The solution was then cooled to 10° C. in an ice-water bath and neutralized to pH of 5.5 by the slow addition of a 7M KOH solution (4.4 L) over 1 hour. The temperature of the solution was maintained below 50° C. The mixture was then diluted with 20% THF in EtOAc (1.8 L) and stirred for 30 minutes. The mixture was then filtered through a pad of Celite filter aid and the filter cake was rinsed with 20% THF in EtOAc (1.8 L). After separation of the layers of the filtrate, the aqueous layer was extracted with 20% THF in EtOAc (1.8 L). The combined organic layer was stirred with MgSO₄ (200 g) and Dacco G-60 charcoals (120 g) for 1 hour. After filtration, the filtrate was concentrated in vacuo to yield brown oil. The crude oil was dissolved in EtOAc (540 mL) and stirred at the room temperature for 20 hours. Beige solids crystallized from the solution. The mixture was further diluted with heptane (700 mL) and stirred for an additional 5 hours at the room temperature. The solid product was collected by vacuum filtration, rinsed with a mixture of EtOAc and heptane (1:4) (700 m), and dried in a vacuum oven at 45° C. for 24 hours. The product was obtained as a beige solid.

mp 162-164° C.

¹H NMR (CDCl₃, 300 MHz) δ7.86 (br s, 2H), 7.04 (m, 2H), 6.76 (s, 1H), 6.73 (m, 2H), 3.16 (m, 1H), 3.05 (m, 1H), 2.80 (dd, J=41.9, 13.6 Hz, 2H), 2.60 (m, 2H), 1.81 (m, 4H)

MS (ESI): m/z 244.1 (MH⁺)

Elemental analysis for C₁₅H₁₇NO₂: Calc'd: C, 74.05; H, 7.04; N, 5.76. Found: C, 73.82; H, 7.33; N, 5.95.

### EXAMPLE 6

### (4R)-1,2,3,5-tetrahydro-spiro[4H-1-benzazepine-4,1′-[2]cyclopentene]-3′-carboxylic acid. (1R)-7,7-dimethyl-2-oxo-bicyclo[2.2.1]heptane-1-methanesulfonic acid salt

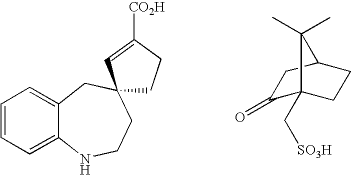

1,2,3,5-tetrahydro-spiro[4H-1-benzazepine-4,1′-[2]cyclopentene]-3′-carboxylic acid (7.02 mol, 1708 g) was suspended in 16 liters of methanol and heated to 65 to 70° C. After 1 h, a solution of (−)-camphorsulfonic acid (5.61 mol, 1304 g) in methanol (10 L) was added over 30 min. The reaction mixture was heated to reflux for an hour. The mixture was then cooled to the room temperature and the product, (4R)-1,2,3,5-tetrahydro-spiro[4H-1-benzazepine-4,1′-[2]cyclopentene]-3′-carboxylic acid (1S)-7,7-dimethyl-2-oxo-bicyclo[2.2.1]heptane-1-methanesulfonic acid salt, was isolated by vacuum filtration. The crude product was purified by recrystallization from methanol (20 L). The product was isolated as off-white to beige solid.

mp 289-291° C.

¹H NMR (DMSO-d₆, 300 MHz) δ7.46 (m, 1H), 7.35 (m, 3H), 6.38 (s, 1H), 3.44 (br m, 1H), 3.30 (br m, 1H), 3.10 (m, 2H), 2.92 (m, 2H), 2.62 (m, 2H), 2.51 (m, 2H), 2.47 (m, 2H), 2.26 (m, 1H), 2.06 (m, 1H), 1.94 (m, 2H), 1.84 (m, 2H), 1.64 (m, 2H), 1.29 (m, 2H), 1.04 (s, 3H), 0.75 (s, 3H)

MS (ESI): m/z 244.1 (MH⁺)

Elemental analysis for C₂₅H₃₃NO₆S: Calc'd: C, 63.13; H, 6.99; N, 2.95; S, 6.74. Found: C, 62.86; H, 6.91; N, 2.77; S, 6.76.

### EXAMPLE 7

### (4R)-1-[4-[([1,1′-biphenyl]-2-ylcarbonyl)amino]benzoyl]-1,2,3,5-tetrahydro-spiro[4H-1-benzazepine-4,1′-[2]cyclopentene]-3′-carboxylic acid

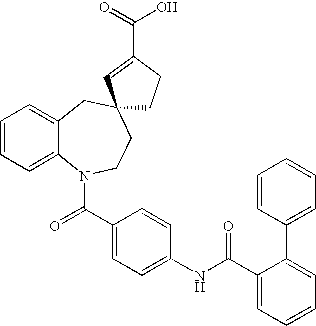

A solution of 4-(2-phenylbenzoyl)aminobenzoic acid (0.105 mol, 33.4 g) in 241 mL of toluene was treated with thionyl chloride (0.21 mol, 25.0 g) and a catalytic amount of DMF (0.009 mol, 0.66 g) at 95° C. for one hour and at 100° C. for an additional hour. Most of the toluene was then removed by distillation (180 ml). The resulting slurry was diluted in dichloromethane (185 mL) to yield the acid chloride slurry to be used in the next step.

In a separated reaction vessel, a solution of (4R)-1,2,3,5-tetrahydro-spiro[4H-1-benzazepine-4,1′-[2]cyclopentene]-3′-carboxylic acid (1S)-7,7-dimethyl-2-oxo-bicyclo-[2.2.1]heptane-1-methanesulfonic acid (0.105 mol, 50.0 g) and pyridine (0.63 mol, 49.9 g) in toluene (172 mL) was treated with chlorotrimethylsilane (0.346 mol, 37.7 g) at 0° C. for one hour. At this temperature the slurry of the acid chloride prepared as above was added to the reaction mixture. After stirred for 20 hours, the reaction mixture was quenched by addition of aqueous hydrochloric acid (16%, 0.4 mol, 83.0 g). Most of the dichloromethane (127 mL) was then removed by distillation at 85° C. and the residue was re-dissolved in DMF (235 mL) at 100° C. At this temperature water (110 mL) was added and the reaction mixture was cooled to the room temperature. The product precipitated and was collected by vacuum filtration. The filter cake was washed with a mixture of ethanol/water (1:1, 86 mL), water (172 mL), and dried in a vacuum oven at 75° C. for 24 h. The product was isolated as an off-white solid.

mp 263-265° C.

¹H NMR (DMSO-d₆, 300 MHz) δ12.3 (s, 1H), 10.3 (s, 1H), 7.54 (m, 2H), 7.44 (m, 2H), 7.33 (m, 7H), 7.06 (m, 4H), 6.66 (br s, 1.6H), 6.23 (br s, 0.4H), 4.76 (br m, 1H), 3.34 (m, 1H), 2.81 (m, 1H), 2.50 (m, 4H), 1.91 (m, 2H), 1.58 (m, 2H)

MS (ESI): m/z 543.4 (MH⁺).

### EXAMPLE 8

### (4R)-1-[4-[([1,1′-biphenyl]-2-ylcarbonyl)amino]benzoyl]-N-[2-(dimethylamino)ethyl]-1,2,3,5-tetrahydro-spiro[4H-1-benzazepine-4,1′-[2]cyclopentene]-3′-carboxamide benzoate

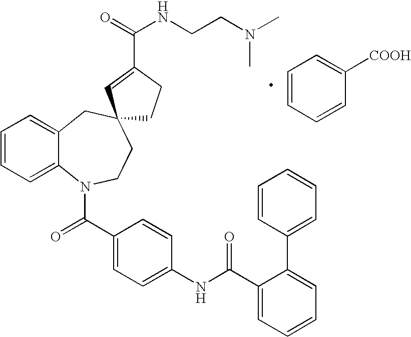

A solution of (4R)-1-[4-[([1,1′-biphenyl]-2-ylcarbonyl)amino]benzoyl]-1,2,3,5-tetrahydro-spiro[4H-1-benzazepine-4,1′-[2]cyclopentene]-3′-carboxylic acid (0.085 mol, 46.12 g) in toluene (460 mL), was treated with thionyl chloride (0.116 mol, 13.81 g). The reaction mixture was heated to 100° C. and stirred for 2 hours. The reaction mixture became a clear solution. Toluene and the excess amount of thionyl chloride were then distilled off. The resulting crude acid chloride was dissolved in a mixture of dichloromethane and EtOAc (1/3 ratio, 400 g) and added to a solution of N,N′-dimethylaminoethylamine (0.105 mol, 9.70 g) and triethylamine (0.092 mol, 9.31 g) in EtOAc (400.0 g) at 0-5° C. The reaction mixture was stirred at 0-5° C. for one hour and then quenched with aqueous hydrochloric acid (10%, 150.0 g). After separation of the layers, the aqueous layer was treated with aqueous sodium hydroxide (27%, 137.0 g) until the pH of the mixture became around 10. The aqueous layer was extracted with EtOAc (500.0 g) and dried with sodium sulfate. About half of the solvent was distilled off to yield a solution of the title product as a free base. To the solution of the free base at 50° C. was added a solution of benzoic acid (0.17 mol, 20.71 g) in EtOAc (300.0 g). The product was precipitated after stirring at 60° C. for 1 hour and 20° C. for 4 hours. The precipitate was then collected by vacuum filtration, and dried in a vacuum oven at 60° C. for 16 hours. The product was isolated as a white solid.

mp 180-185° C. (decomp.)

¹H NMR (DMSO-d₆, 300 MHz) δ10.30 (s, 1H), 7.94 (d, J=7.0 Hz, 2H), 7.80 (m, 1H), 7.57 (m, 3H), 7.47 (m, 4H), 7.33 (m, 8H), 7.11 (m, 4H), 6.67 (br t, J=8.0 Hz, 1H), 6.44 (br s, 0.6H), 6.10 (br s, 0.4H), 4.68 (br m, 1H), 3.26 (m, 3H), 2.82 (m, 1H), 2.50 (s, 2H), 2.46 (m, 4H), 2.26 (s, 3.6H), 2.19 (s, 2.4H), 1.89 (m, 2H), 1.54 (m, 2H)

MS (ESI): m/z 613.0 (MH⁺)

Elemental analysis for C₄₆H₄₆N₄O₅: Calc'd: C, 75.18; H, 6.31; N, 7.62. Found: C, 74.92; H, 6.19; N, 7.43.

### EXAMPLE 9

### 4-[(Bi phenyl-2-carbonyl)-amino]-benzoyl chloride

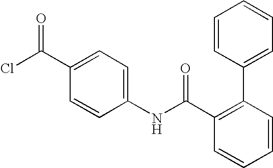

A mixture of 2-biphenylcarboxylic acid (25 g, 0.13 mol) in thionyl chloride (80 mL) was stirred at room temperature overnight and the excess SOCl₂ removed under reduced pressure to yield biphenyl-2-carbonyl chloride as a yellow oil. The oil was dissolved in methylene chloride (60 mL) and slowly added through an addition funnel to a solution of methyl 4-aminobenzoate (20 g, 0.13 mol) and triethylamine (28 mL, 0.198 mol) dissolved in methylene chloride (400 mL). The resulting mixture was stirred at room temperature for 4-5 hours and water (500 mL) was added. The layers were separated; the CH₂Cl₂ layer dried over MgSO₄ and the solvent removed under reduced pressure. The resulting solids were washed with diethyl ether and dried to yield methyl 4-[(biphenyl-2-carbonyl)-amino]-benzoate as a tan solid.

m.p. 160-161° C.

To a stirred solution of the methyl 4-[(biphenyl-2-carbonyl)-amino]-benzoate (37 g, 0.11 M) in CH₃OH (400 mL) was slowly added 6.6N NaOH (100 mL). Stirring was continued till all solids dissolved (about 6 hours). The methanol was removed under reduced pressure, the solids dissolved in H₂O and concentrated HCl was slowly added to the stirred solution. The mixture was stirred at room temperature overnight and the resulting white solid precipitates collected and dried to yield 4-[(biphenyl-2-carbonyl)-amino]-benzoic acid as a white crystalline solid.

MS: m/z (M+) 318

The 4-[(biphenyl-2-carbonyl)-amino]-benzoic acid (34 g, 0.1 M) was stirred in thionyl chloride (260 mL) with gentle heating (40° C., oil bath) for 4 hours. The residual semi solid was diluted with toluene and filtered to yield the title product as a white solid.

m.p. 148-150° C.

### EXAMPLE 10

### (1′R)-N-[2-(Dimethylamino)ethyl]-1-[4-[(2-fluorobenzoyl)amino]benzoyl]-1,2,3,5-tetrahydrospiro[4H-1-benzazepine-4,1′-[2]cyclopentene]-3′-carboxamide phosphate

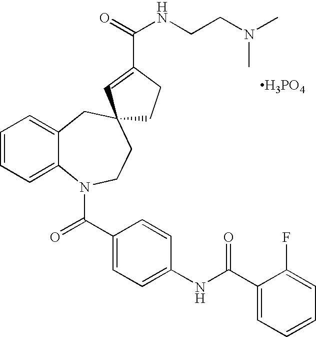

Step A:

4-Amino benzoic acid (20.0 g, 0.146 mol) was suspended in toluene at the room temperature. Pyridine (50.76 g, 0.646 mol) was added and the mixture was stirred at the room temperature for 15 minutes. The reaction mixture was then cooled to 0° C. and chlorotrimethylsilane (98.0 g, 2.2 mol) was added. The reaction mixture was stirred at 0° C. for 30 minutes. A solution of 2-fluorobenzoylchloride (23.14 g, 0.146 mol) in toluene was then added within 15 minutes. The resulting mixture was stirred at 0° C. for 1.5 hours. The reaction was quenched with aqueous hydrochloric acid and diluted with ethanol (100 mL). After stirring at the room temperature for 15 minutes, the mixture was heated to 85° C. for 30 minutes. The reaction mixture was then cooled to the room temperature, resulting in the precipitation of a white sold. The solid product was isolated by vacuum filtration, washed with water and ethanol, and dried in a vacuum oven to yield 4-(2-fluoro-benzoylamino)-benzoic acid as a white solid.

The solid 4-(2-fluoro-benzoylamino)-benzoic acid prepared as above (127.22 g, 0.105 mol) was suspended in toluene (650 mL) at the room temperature. Thionyl chloride (1.5 mol eq.) and a catalytic amount of DMF were added and the mixture was heated to 100° C. for 1 h until the suspension became a clear solution. The reaction solution was concentrated in vacuum to yield 4-(2-fluoro-benzoylamino)-benzoyl chloride, which was diluted in dichloromethane (350 mL) for the next step.

Step B:

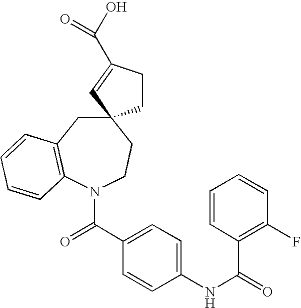

In a separated reaction vessel, the (1R)-(−)-camphorsulfonic acid salt of 1,2,3,5-tetrahydro-spiro[4H-1-benzazepine-4,1′-[2]cyclopentene]-3′-carboxylic acid (50.0 g, 0.105 mol) was suspended in toluene (450 mL) at the room temperature. Pyridine (6.0 mol eq.) was added and the mixture stirred at the room temperature for 30 min. The mixture was then cooled to 0° C. and chlorotrimethylsilane (3.3 mol eq.) was added. The mixture was stirred at 0° C. for 30 minutes. The 4-(2-fluoro-benzoylamino)-benzoyl chloride prepared above (1.0 mol eq.) was added within 15 minutes. The resulting mixture was stirred at 0° C. for 2 hours and then quenched with aqueous hydrochloric acid (32-34%. 40.0 g, 0.42 mol). The solvents were distilled off at 65° C. During distillation, the product precipitated as a white solid. After cooling to the room temperature, the resulting solid product, (4R)-1-[4-[(2-fluorobenzoyl)amino]benzoyl]-1,2,3,4-tetrahydro-spiro[4H-1-benzazepine-4,1′-[2]cyclopentene]-3′-carboxylic acid, was isolated by vacuum filtration, washed with water, recrystallized from ethanol/H₂O.

Step C:

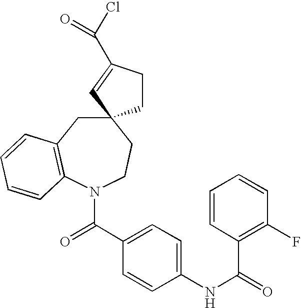

The solid product prepared as in Step B above (40.0 g, 0.082 mol) was suspended in toluene (350 mL) at room temperature. Thionyl chloride (2.0 mol eq.) and a catalytic amount of DMF were added and the mixture was heated to 100° C. for 1 hour until the suspension became a clear solution. The solvent was then removed in vacuum to yield the corresponding acid chloride product, which was diluted in EtOAc (400 mL) and used in the subsequent steps without further purification.

Step D:

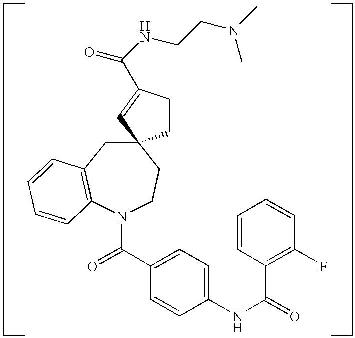

In a separated reaction vessel, N,N′-dimethylethylendiamine (8.72 g, 0.098 mol) was dissolved in EtOAc (250 mL) at the room temperature. Triethylamine (1.3 mol eq.) was added and the mixture was cooled to 0° C. and stirred for 30 minutes. The solution of acid chloride prepared as in Step C above (1.0 mol eq.) in EtOAc was added within 15 minutes. The resulting mixture was stirred at 0° C. for 1.5 hours and then quenched with aqueous hydrochloric acid. The layers were separated and the organic layer was extracted once with aqueous hydrochloric acid (32-34%, 8.98 g, 0.246 mol). The combined aqueous layer was treated with aqueous sodium hydroxide solution until the pH value of the mixture became larger than 10. The aqueous layer was extracted twice with EtOAc (200 mL). The combined organic layer was dried over sodium sulfate (50.0 g). After filtration and concentration, the corresponding crude product, (4R)-1-[4-[(2-fluorobenzoyl)amino]benzoyl]-N-[2-(dimethylamino)ethyl]-1,2,3,4-tetrahydro-spiro[4H-1-benzazepine-4,1′-[2]cyclopentene]-3′-carboxamide, was obtained as an off-white solid.

Step E:

The crude amine product prepared as in Step D above (8.12 g, 0.016 mol) was dissolved in methanol (100 mL) and heated to 70° C. Water (˜1.5 mol eq.) was added. A solution of phosphoric acid (0.9 mol eq., based on the assay of the crude product) in methanol was added at 70° C. within 30 minutes, followed by the addition of methyl tert-butyl ether (200 mL) until the solution became cloudy. The mixture was cooled to the room temperature and the salt of the title compound (4R)-1-[4-[(2-fluorobenzoyl)amino]benzoyl]-N-[2-(dimethylamino)ethyl]-1,2,3,4-tetrahydro-spiro[4H-1-benzazepine-4,1′-[2]cyclopentene]-3′-carboxamide phosphate was precipitated slowly and isolated by vacuum filtration after 16 hours as a light beige solid.

mp 148-158° C. (decomp.)

¹H NMR (DMSO-d₆, 300 MHz) δ10.50 (s, 1H), 8.1 (br m, 1H), 7.56 (m, 4H), 7.33 (m, 3H), 7.12 (m, 5H), 6.69 (m, 1H), 6.54 (s, 0.6H), 6.14 (s, 0.4H), 4.73 (m, 1H), 3.35 (m, 3H), 2.80 (m, 3H), 2.59 (m, 3H), 2.52 (s, 3.6H), 2.45 (m, 2.4H), 1.91 (m, 2H), 1.55 (m, 2H)

MS (ESI): m/z 555.4 (MH⁺)

Elemental analysis for C₃₃H₃₅FN₄O₃.H₃PO₄: Calc'd: C, 60.73; H, 5.87; F, 2.91; N, 8.58; P, 4.75. Found: C, 59.82; H, 6.13; F, 2.94; N, 8.54; P, 4.57.

### EXAMPLE 11

### 3-Methoxy-4-nitro-benzoic acid methyl ester

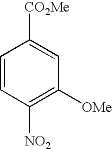

A 5-L, 3-necked, round-bottomed flask fitted with an overhead stirrer and a 250 mL addition funnel was charged with 3-hydroxy-4-nitrobenzoic acid (122 g, 0.66 mol), acetone (reagent grade, 1.5 L) and powdered K₂CO₃ (185 g). To this stirred suspension dimethylsulfate (127 mL) was added drop-wise. The suspension was stirred at room temperature for 18 h and filtered. The filtrate was concentrated under reduced pressure to about half the volume (ca 750 mL), transferred to a 3-L beaker and water (1-L) was added with stirring. The precipitated product was collected by filtration and dried in vacuum to obtain the title compound as a white crystalline solid.

mp 87-88° C.

MS (M+H)⁺=212.1

¹H NMR (400 MHz, CDCl₃) δ: 7.77 (d, J=8.3, 1H), 7.68 (d, J=1.5 Hz, 1H), 7.63 (d, J=8.3 Hz, 1H), 3.94 (s, 3H), 3.90 (s, 3H).

### EXAMPLE 12

### 4-Amino-3-methoxy-benzoic acid methyl ester

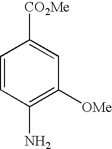

To a 2-L, Parr high-pressure hydrogenation bottle (glass rated to 80 psi) was charged Pd/C (10 wt % on Carbon, 5 g), EtOAc (800 mL) and 3-methoxy-4-nitrobenzoic acid methyl ester (120.5 g, 0.57 mol). The reaction mixture was charged with H₂ (30 psi) on a Parr apparatus. Charging H₂ was continued carefully several times until the pressure remained steady. This took approximately about 3 h. The reaction was shaken for an additional 0.5 h. After the hydrogenation, the reaction mixture was diluted with ethyl acetate to dissolve some of the precipitated product and directly passed through a short pad of Celite, and washed with EtOAc. The solvent was evaporated to yield 4-amino-3-methoxy-benzoic acid methyl ester as a white solid.

MS (electro spray, positive mode), (M+H)⁺ 182.1.

¹H NMR (400 MHz, CDCl₃) δ: 7.55 (dd, J=8.1, 1.7 Hz, 1H), 7.45 (d, J=1.7 Hz, 1H), 6.66 (d, J=8.1 Hz, 1H), 4.21 (s, 2H), 3.90 (s, 3H), 3.86 (s, 3H).

### EXAMPLE 13

### 4-(2-Chloro-5-fluoro-benzoylamino)-3-methoxy-benzoic acid methyl ester

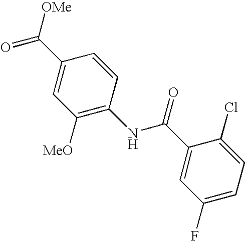

A dry, 3-L, 3-necked, round-bottomed flask equipped with a thermometer and addition funnel was charged a solution of 4-amino-3-methoxy-benzoic acid methyl ester (96 g, 0.53 mol, 1.0 equiv.) and Et₃N (88 ml, 0.64 mol, 1.2 equiv.) in CH₂Cl₂ (1.2 L). The solution was cooled to 0° C. by an ice bath and 2-chloro-5-fluoro-benzoyl chloride (110 g, 0.57 mol, 1.05 equiv.) was added drop-wise over 40 min at 0° C. After the addition, the reaction mixture was stirred at 0° C. for further 1.5 h. The organic layer was washed with brine three times, dried over MgSO₄, filtered, and evaporated to yield 4-(2-chloro-5-fluoro-benzoylamino)-3-methoxy-benzoic acid methyl ester as a white solid.

MS (electro spray, positive mode), (M+H)⁺ 338.0.

¹H NMR (400 MHz, CDCl₃) δ: 8.84 (s, 1H), 8.61 (d, J=8.4 Hz, 1H), 7.75 (dd, J=8.4, 1.7 Hz, 1H), 7.60 (d, J=1.7 Hz, 1H), 7.55 (dd, J=8.4, 3.0 Hz, 1H), 7.45 (dd, J=8.4, 4.8 Hz, 1H), 7.20-7.13 (m, 1H), 3.97 (s, 3H), 3.93 (s, 3H)

### EXAMPLE 14

### 4-(2-Chloro-5-fluoro-benzoylamino)-3-methoxy-benzoic acid

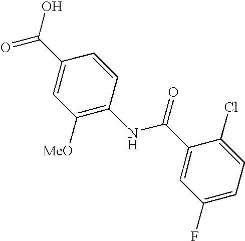

LiOH (14.1 g, 0.59 mol, 1.1 equiv.) dissolved in H₂O (200 mL) was added drop-wise over 45 minutes to a solution of 4-(2-chloro-5-fluoro-benzoylamino)-3-methoxy-benzoic acid methyl ester (180 g, 0.53 mol, 1 equiv.) in THF (1800 mL) at room temperature. The reaction mixture was stirred at room temperature for 16 h. The solvent was evaporated under reduced pressure and the residue re-dissolved in water (ca. 3 L). The insoluble solid was filtered off. Under vigorous stirring, the aqueous filtrate solution was acidified with concentrated HCl aqueous solution (37%) until pH<2. The resulting white solid precipitate was filtered and washed with water. The wet filter cake was then transferred to a flask and dried on rotary evaporator under vacuum at 50° C. overnight to yield 4-(2-chloro-5-fluoro-benzoylamino)-3-methoxy-benzoic acid as a dry, fine white powder.

MS (electro spray, negative mode), (M−H)⁻ 322.0.

¹H NMR (400 MHz, DMSO-d₆) δ: 12.90 (s, 1H), 10.01 (s, 1H), 8.22 (d, J=8.3 Hz, 1H), 7.65-7.45 (m, 4H), 7.45-7.30 (m, 1H), 3.88 (s, 1H)

### EXAMPLE 15

### 4-(2-Chloro-5-fluoro-benzoylamino)-3-methoxy-benzoyl chloride

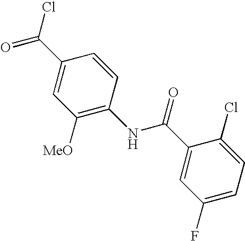

4-(2-chloro-5-fluoro-benzoylamino)-3-methoxy-benzoic acid (152 g, 0.45 mol, 1 equiv.) was suspended in CH₂Cl₂ (1.5 L) and DMF (1 mL) was added. Oxalyl chloride (71.6 g, 0.56 mol, 1.2 equiv.) was added drop-wise over 30 minutes at 0° C. After addition, the cold bath was removed and the reaction mixture was further stirred at room temperature for 3.5 h. The solvent and any unreacted oxalyl chloride were evaporated to yield a white solid, which was further dried on a rotary evaporator under vacuum at 40° C. overnight to yield dry 4-(2-chloro-5-fluoro-benzoylamino)-3-methoxy-benzoyl chloride as a white solid.

The title compound was isolated substantially free of byproducts that have the ortho and/or para positions to the methoxy group halogenated. The terms “substantially free of byproducts” in this context mean that such byproducts are not detectable by HPLC.

MS (electro spray, negative mode), (M−H)⁻ 339.9

¹H NMR (400 MHz, CDCl₃) δ: 8.97 (s, 1H), 8.71 (d, J=8.6 Hz, 1H), 7.91 (dd, J=8.6, 1.9 Hz, 1H), 7.60 (d, J=1.9 Hz, 1H), 7.57 (dd, J=8.6, 3.1 Hz, 1H), 7.47 (dd, J=8.6, 4.8 Hz, 1H), 7.21-7.15 (m, 1H), 3.99 (s, 3H).

### EXAMPLE 16

### (4R)-1,2,3,5-tetrahydro-spiro[4H-1-benzazepine-4,1′-[2]cyclopentene]-3′-carboxylic acid

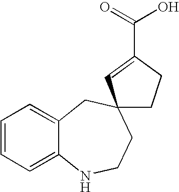

In a 3-necked, 5-L, round-bottomed flask fitted with an air-pump stirrer, (4R)-2,3,4,5-tetrahydrobenzazepine-4-spiro-3′-cyclopent-1′-ene-carboxylicacid-(1R,4S)-7,7-dimethyl-2-oxo-bicyclo[2.2.1]heptane-methanesulfonate (500 g, 1.05 mol) was suspended in H₂O (2 L) to yield a reaction mixture with a pH of about 3-4. With an addition funnel, saturated aqueous NaHCO₃ solution was added slowly to the mixture until pH 6. CH₂Cl₂ (1 L) was then added and the slurry mixture stirred for 1 h. Any remaining starting material in the mixture was then filtered off. The layers were separated and the aqueous layer extracted with CH₂Cl₂ (2×150 mL). The combined organic layer was dried with Na₂SO₄, filtered and concentrated to yield (4R)-1,2,3,5-tetrahydro-spiro[4H-1-benzazepine-4,1′-[2]cylopentene]-3′-carboxylic acid as a dark gray solid.

To the remaining starting material, the process was repeated again until all the salts were completely converted to free acid.

All of crude (4R)-1,2,3,5-tetrahydro-spiro[4H-1-benzazepine-4,1′-[2]cylopentene]-3′-carboxylic acid was combined, suspended in EtOAc/hexanes (1:1) stirring overnight at room temperature and then filtered to yield (4R)-1,2,3,5-tetrahydro-spiro[4H-1-benzazepine-4,1′-[2]cylopentene]-3′-carboxylic acid as a gray solid in 88% yield.

MS (electro spray, positive mode), (M+H)⁺ 244.1.0.

¹H NMR (400 MHz, CDCl₃) δ: 7.09-7.01 (m, 2H), 6.76 (t, J=6.3 Hz, 1H), 6.77 (s, 1H), 6.72 (d, J=7.6 Hz, 1H), 3.17-3.14 (m, 1H), 3.07-3.05 (m, 1H), 2.82 (dd, J=53.3, 13.64 Hz, 2H), 2.71-2.54 (m, 2H), 1.92-1.68 (m, 4H).

### EXAMPLE 17

### (4R)-1,2,3,5-tetrahydro-spiro[4H-1-benzazepine-4,1′-[2]cylopentene]-3′-carboxylic acid ethyl ester

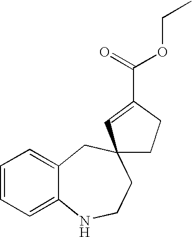

In a 3-necked, 3-L, round-bottomed flask fitted with an inlet thermometer and air-pump stirrer, (4R)-1,2,3,5-tetrahydro-spiro[4H-1-benzazepine-4,1′-[2]cylopentene]-3′-carboxylic acid (225.0 g, 0.92 mol) was slurried in EtOH (1 L). The flask was chilled in an ice bath and slowly, conc. H₂SO₄ (90 g) was added while maintaining the internal temperature between 15 and 25° C. The ice bath was removed after the addition was complete and the reaction was stirred overnight at room temperature. The reaction was 98% complete after the reaction mixture was heated for another 5 days at 40° C. The reaction mixture was concentrated to a black oil, diluted in CH₂Cl₂ (1 L), then washed with H₂O (2×500 mL), saturated NaHCO₃ solution (1×1 L) and saturated NaCl solution (1×1 L). The extracted organic layer was dried with Na₂SO₄, filtered and concentrated to yield (4R)-1,2,3,5-tetrahydro-spiro[4H-1-benzazepine-4,1′-[2]cylopentene]-3′-carboxylic acid ethyl ester as a black oil. Crude (4R)-1,2,3,5-tetrahydro-spiro[4H-1-benzazepine-4,1′-[2]cylopentene]-3′-carboxylic acid ethyl ester was purified by filtration chromatography (silica gel column: 14 cm OD, 8 cm in height and eluting with 4/1 hexanes/EtOAc). The desired fractions were combined to recover (4R)-1,2,3,5-tetrahydro-spiro[4H-1-benzazepine-4,1′-[2]cylopentene]-3′-carboxylic acid ethyl ester as dark red oil. Filtration chromatography was repeated again and fractions containing the product were combined to yield (4R)-1,2,3,5-tetrahydro-spiro[4H-1-benzazepine-4,1′-[2]cylopentene]-3′-carboxylic acid ethyl ester as a yellow oil.

MS (electro spray, positive mode), (M+H)⁺ 272.1.

¹H NMR (400 MHz, CDCl₃) δ: 7.08-7.01 (m, 2H), 6.83 (t, J=7.3 Hz, 1H), 6.71 (d, J=7.8 Hz, 1H), 6.63 (t, J=2.0 Hz, 1H), 4.18 (dd, J=14.4, 7.3 Hz, 2H), 3.77 (br s, 1H), 3.19-3.13 (m, 1H), 3.07-3.0 (m, 1H), 2.81 (dd, J=56.6, 13.6 Hz, 2H), 2.70-2.53 (m, 2H), 1.91-1.65 (m, 4H), 1.29 (t, J=7.1 Hz, 3H).

### EXAMPLE 18

### (4R)-1-[4-(2-chloro-5-fluorobenzoyl)amino-3-methoxybenzoyl]-1,2,3,5-tetrahydro{4H-1-benzazepine-4,1′-[2]cyclopentene]-3′-carboxylic acid ethyl ester

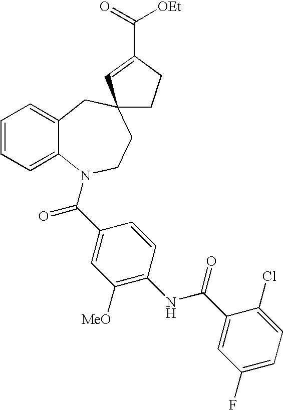

In a dried, 1-neck, 3-L, round-bottomed flask fitted with an air-pump stirrer, combined ester (4R)-1,2,3,5-tetrahydro{4H-1-benzazepine-4,1′-[2]cyclopentene]-3′-carboxylic acid ethyl ester (105 g, 0.39 mol) and 4-(2-chloro-5-fluoro-benzoyl)amino-3-methoxy-benzoyl chloride (146 g, 0.43 mol) in CH₂Cl₂ (1 L). The reaction mixture (suspension) was chilled using an ice bath to 0° C. and triethylamine (65 mL, 0.47 mol, 1.2 eq) was added slowly during a period of 15 minutes. The ice bath was removed and reaction mixture allowed to warm up-to room temperature. After 30 minutes HPLC analysis indicated the reaction was complete. The reaction mixture was quenched with H₂O (500 mL) and the layers separated. The organic layer was washed with saturated NaHCO₃ solution (1×500 mL) and saturated NaCl solution (1×500 mL). The extracted organic layer was dried with Na₂SO₄ and filtered. The filtrate containing crude product was concentrated to oil and purified by filtration chromatography (silica gel column: 14 cm OD, 8 cm in height and eluting with 4/1 EtOAc/hexanes). The desired fractions were combined yield (4R)-1-[4-(2-chloro-5-fluorobenzoyl)amino-3-methoxybenzoyl]-1,2,3,5-tetrahydro{4H-1-benzazepine-4,1′-[2]cyclopentene]-3′-carboxylic acid ethyl ester as an orange oil.

MS (electro spray, negative mode), (M+H)⁺ 577.0.

¹H NMR (400 MHz, CDCl₃) δ: 8.66 (s, 1H), 8.26 (d, J=8.3 Hz, 1H), 7.48 (dd, J=8.6, 3.0, 1H), 7.41 (dd, J=8.6, 4.5 Hz, 1H), 7.22-7.09 (m, 3H), 7.0 (t, J=7.0 Hz, 1H), 6.94 (s, 1H), 6.75-6.67 (m, 2H), 4.84 (bd, J=48 Hz, 1H), 4.25-4.14 (m, 2H), 3.72 (s, 3H), 3.33 (dd, J=13.4, 4.5 Hz, 1H), 3.16-2.96 (m, 1H), 2.75-2.61 (m, 3H), 2.13-1.93 (m, 2H), 1.79-1.72 (m, 3H), 1.34-1.22 (m, 3H).

### EXAMPLE 19

### (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid

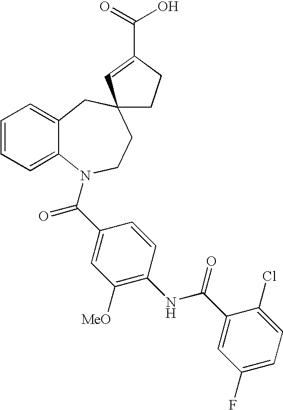

In a 1-necked, 2-L, round-bottomed flask fitted with a magnetic stir bar, (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid ethyl ester (220.0 g, 0.38 mol) was diluted in EtOH/THF (350 mL/350 mL). A hot (ca. 60-70° C.) solution of LiOH (13.7 g, 0.57 mol) in H₂O (200 mL) was slowly added drop-wise to solution over a period of 15 minutes. The reaction mixture was stirred and allowed to cool to room temperature overnight. The reaction mixture was concentrated to an oil, treated with H₂O (1 L), transferred to a separatory funnel and washed with EtOAc (1×500 mL). The aqueous layer was acidified to pH 1-2 using 3 M HCl then extracted with EtOAc (2×500 mL). The extracted organic layer was dried with Na₂SO₄, filtered and concentrated under reduced pressure until precipitation developed in the flask. The precipitated solids were treated with Et₂O/hexanes (600 mL/200 mL) and stirred for 2 h and then filtered. The filtered solids were dried in a high vacuum pump overnight in a rotovap at 60° C. to yield the title compound (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid as a white solid.

mp 178-180° C.

MS (electro spray, negative mode), (M⁺+Na) 571.0

¹H NMR (400 MHz, CDCl₃) δ: 8.66 (s, 1H), 8.26 (d, J=8.3 Hz, 1H), 7.48 (dd, J=8.6, 3.3 Hz, 1H), 7.41 (dd, J=8.8, 4.8 Hz, 1H), 7.23-7.1 (m, 3H), 7.0 (t, J=7.8 Hz, 1H), 6.73-6.67 (m, 2H), 4.86 (bd, J=49.7 Hz, 1H), 3.73 (s, 3H), 3.35 (dd, J=13.6, 5.0 Hz, 1H), 3.15-2.96 (m, 1H), 2.76-2.62 (m, 3H), 2.15-2.0 (m, 2H), 1.82-1.54 (m, 2H)

### EXAMPLE 20

### 1-[4-[([1,1′-Biphenyl]-2-ylcarbonyl)amino]benzoyl]-N-[2-(dimethylamino)ethyl]-1,2,3,5-tetrahydro-2′-oxospiro[4H-1-benzazepine-4,1′-cyclopentane]-3′-carboxamide

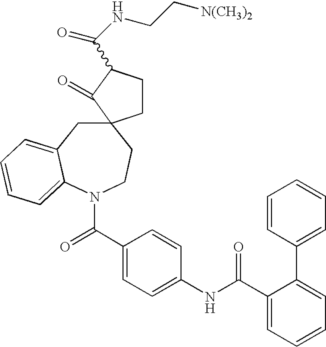

Step A: 4-(3-Ethoxycarbonyl-propyl)-5-oxo-1-(toluene-4-sulfonyl)-2,3,4,5-tetrahydro-1H-benzo[b]azepine-4-carboxylic acid ethyl ester

To a solution of ethyl 2,3,4,5-tetrahydro-1-[(4-methylphenyl)sulfonyl]-5-oxo-1H-1-benzazepine-4-carboxylate (4.67 g, 12.1 mmol; CAS 54620-98-3; U.S. Pat. No. 6,369,110; G. R. Proctor et al., J. Chem. Soc. Perkin Trans 1 1972, 14, 1803-1808) in DMF (24 mL) was added K₂CO₃ (25.0 g, 18.1 mmol). The resulting suspension was treated with ethyl 4-bromobutyrate (1.90 mL, 13.3 mmol) while stirring mechanically under a nitrogen atmosphere at room temperature. After 18 hours, the reaction mixture was diluted with ethyl acetate (50 mL) and quenched by the addition of aqueous 1 N HCl (30 mL). The resulting layers were separated and the organic layer was extracted sequentially with saturated aqueous NaHCO₃, water, and brine. The organic extract was dried over anhydrous MgSO₄ and concentrated in vacuo. The residue was purified via column chromatography on silica gel eluting with ethyl acetate/hexanes (3:7) to yield 4-(3-ethoxycarbonyl-propyl)-5-oxo-1-(toluene-4-sulfonyl)-2,3,4,5-tetrahydro-1H-benzo[b]azepine-4-carboxylic acid ethyl ester as an oil.

Step B: 4-(3-Ethoxycarbonyl-propyl)-1-(toluene-4-sulfonyl)-2,3,4,5-tetrahydro-1H-benzo[b]azepine-4-carboxylic acid ethyl ester

The compound prepared as in STEP A above, (4.76 g, 9.48 mmol) was dissolved in dry 1,2-dichloroethane (45 mL), cooled to 5° C., and then treated with trifluoroacetic acid (1.3 mL), BF₃.Et₂O (1.4 mL), anhydrous methanesulfonic acid (3.2 mL) and triethylsilane (5.7 mL). The reaction was allowed to slowly warm to room temperature over 18 hours. The reaction was cooled to 5° C. and cautiously quenched with saturated aqueous NaHCO₃ (100 mL). The reaction mixture was extracted with ethyl acetate (100 mL) and the ethyl acetate extract was extracted with saturated aqueous NaHCO₃, water, brine (2×), dried over Na₂SO₄, and concentrated in vacuo to yield an oil. The oil was purified by column chromatography on silica gel eluting with hexane/ethyl acetate (17:3) to yield 4-(3-ethoxycarbonyl-propyl)-1-(toluene-4-sulfonyl)-2,3,4,5-tetrahydro-1H-benzo[b]azepine-4-carboxylic acid ethyl ester as a colorless oil.

Step C: Ethyl 1-(4-methylphenyl)sulfonyl)-1,2,3,4-tetrahydro-2′-oxospiro[4H-1-benzazepine-4,1′-cylcopentane]-3′-carboxylate

The compound prepared as in STEP B above, (2.43 g, 4.98 mmol) was dissolved in toluene (25 mL) and treated with potassium tert-butoxide (0.843 g, 7.52 mmol) at room temperature. After 1 hour, the reaction mixture was quenched with aqueous 0.5 N HCl (30 mL) and extracted with ethyl acetate (30 mL). The ethyl acetate extract was washed twice with water, saturated aqueous NaHCO₃, water, brine, dried over anhydrous Na₂SO₄ and concentrate in vacuo. The residue was purified via column chromatography on silica gel eluting with hexane/ethyl acetate (4:1) to yield ethyl 1-(4-methylphenyl)sulfonyl)-1,2,3,4-tetrahydro-2′-oxospiro[4H-1-benzazepine-4,1′-cylcopentane]-3′-carboxylate as a solid.

Step D: Ethyl 1-(4-methylphenyl)sulfonyl)-1,2,3,4-tetrahydro-2′-hydroxyspiro[4H-1-benzazepine-4,1′-cylcopentane]-3′-carboxylate

The compound prepared as in STEP C above, (2.10 g, 5.0 mmol) was dissolved in ethanol (20 mL) at room temperature and treated with NaBH₄ (0.19 g, 5.0 mmol), gas evolution was observed. The resulting suspension was stirred for 30 minutes, concentrated in vacuo, and diluted with ethyl acetate. The resulting suspension was cautiously quenched by the addition of aqueous 1 N HCl (30 mL) and stirred for 5 minutes. The layers were separated and the organic layer was extracted with saturated aqueous NaHCO₃, water, brine, dried over anhydrous MgSO₄, and concentrated in vacuo to yield ethyl 1-(4-methylphenyl)sulfonyl)-1,2,3,4-tetrahydro-2′-hydroxyspiro[4H-1-benzazepine-4,1′-cylcopentane]-3′-carboxylate as a white solid.

Step E: Ethyl 1-(4-methylphenyl)sulfonyl)-1,2,3,4-tetrahydro-2′-(tert-butyldimethylsilyloxy)spiro[4H-1-benzazepine-4,1′-cylcopentane]-3′-carboxylate

The compound prepared as in STEP D above (0.415 g, 0.94 mmol) was combined with 2,6-lutidene (0.657 mL, 5.64 mmol) in dry dichloromethane (10 mL) and treated with tert-butyidimethylsilyl trifluoromethanesulfonate (0.648 mL, 2.82 mmol) while stirring under an argon atmosphere at room temperature. After 20 minutes the reaction was quenched by the addition of saturated aqueous NaHCO₃. The layers were separated and the organic layer was extracted with saturated aqueous NaHCO₃, brine, dried over MgSO₄, and concentrated in vacuo. The residue was purified via column chromatography on silica gel eluting with hexane/ethyl acetate (9:1) to yield ethyl 1-(4-methyl phenyl)sulfonyl)-1,2,3,4-tetrahydro-2′-(tert-butyldimethylsilyloxy)spiro[4H-1-benzazepine-4,1′-cylcopentane]-3′-carboxylate as a syrup.

Step F: Ethyl 1,2,3,4-tetrahydro-2′-(tert-butyldimethylsilyloxy)spiro[4H-1-benzazepine-4,1′-cylcopentane]-3′-carboxylate

The compound prepared as in STEP E above (0.309 g, 0.55 mmol) was dissolved in anhydrous methanol (10 mL) and combined with magnesium turnings (0.267 g, 11 mmol) in a sealed tube and heated while magnetically stirring at 65° C. over 18 hours. The reaction was cooled to room temperature, filtered through filter agent, and concentrated in vacuo. The residue was triturated 3 times with ethyl acetate and the combined ethyl acetate triturations filtered through filter agent. The filtrate was extracted twice with brine, dried over anhydrous Na₂SO₄, and concentrated in vacuo to yield ethyl 1,2,3,4-tetrahydro-2′-(tert-butyldimethylsilyloxy)spiro[4H-1-benzazepine-4,1′-cylcopentane]-3′-carboxylate as a syrup.

Step G: Ethyl 1-[4-[([1,1′biphenyl]-2-ylcarbonyl)amino]benzoyl]-1,2,3,4-tetrahydro-2′-(tert-butyldimethylsilyloxy)spiro[4H-1-benzazepine-4,1′-cylcopentane]-3′-carboxylate

To a slurry of 4-[([1,1′-biphenyl]-2-ylcarbonyl)amino]benzoic acid (0.187 g, 0.59 mmol; CAS 168626-74-2; WO 0132639) in dichloromethane (2 mL) was added was added N,N-dimethylformamide (0.02 mL), thionyl chloride (0.086 mL, 1.18 mmol) while stirring at room temperature under an argon atmosphere. After 18 hours, the resulting solution was concentrated in vacuo, dissolved in dry toluene, concentrated in vacuo, and dissolved in dichloromethane (2 mL). The resulting solution of acid chloride was added dropwise to a solution of the compound prepared as in STEP F above (0.19 g, 0.47 mmol), triethylamine (0.41 mL, 2.95 mmol), and N,N-dimethylformamide (0.02 mL) in dichloromethane (2 mL) while stirring at room temperature. After 72 hours, the reaction mixture was quenched with saturated aqueous NaHCO₃ and extracted with ethyl acetate. The ethyl acetate layer was extracted with saturated aqueous NaHCO₃, brine, dried over anhydrous Na₂SO₄, and concentrated in vacuo. The residue was purified via column chromatography on silica gel eluting with hexane/ethyl acetate (7:3) to yield ethyl 1-[4-[([1,1′biphenyl]-2-ylcarbonyl)amino]benzoyl]-1,2,3,4-tetrahydro-2′-(tert-butyldimethylsilyloxy)spiro[4H-1-benzazepine-4,1′-cylcopentane]-3′-carboxylate as a glass.

Step H: 1-[4-[([1,1′biphenyl]-2-ylcarbonyl)amino]benzoyl]-1,2,3,4-tetrahydro-21-(tert-butyidimethylsilyloxy)spiro[4H-1-benzazepine-4,1′-cylcopentane]-3′-carboxylic acid

The compound prepared as in STEP G above (0.250 g, 0.36 mmol) was combined with potassium hydroxide (0.071 g, 1.08 mmol) in ethanol (20 mL) at heated at reflux while stirring under a nitrogen atmosphere. After 18 hours, the reaction mixture was concentrated in vacuo, dissolved in water, adjusted to pH 2 with aqueous 1 N HCl, and extracted with ethyl acetate. The ethyl acetate layer was washed with brine, dried over anhydrous Na₂SO₄, and concentrated in vacuo to yield 1-[4-[([1,1′biphenyl]-2-ylcarbonyl)amino]benzoyl]-1,2,3,4-tetrahydro-2′-(tert-butyidimethylsilyloxy)spiro[4H-1-benzazepine-4,1′-cylcopentane]-3′-carboxylic acid as a solid.

Step I: 1-[4-[([1,1′biphenyl]-2-ylcarbonyl)amino]benzoyl]-N-[2-(dimethylamino)ethyl]-1,2,3,4-tetrahydro-2′-(tert-butyldimethylsilyloxy)spiro[4H-1-benzazepine-4,1′-cylcopentane]-3′-carboxamide

The compound prepared as in STEP H above (0.234 g, 0.35 mmol) and N-methylmorpholine (0.106 mL, 0.97 mmol) were dissolved in dry tetrahydrofuran (5 mL) and treated dropwise with isopropylchloroformate (0.048 mL, 0.37 mmol) while stirring under a nitrogen atmosphere at room temperature. After 30 minutes, a solution of N,N-dimethylethylene-diamine (0.062 mL, 0.58 mmol) and N-methylmorpholine (0.106 mL, 0.97 mmol) in tetrahydrofuran (0.5 mL) was added and the reaction mixture was stirred for 1 hour. The reaction was quenched via the addition of water and extracted twice with ethyl acetate. The combined ethyl acetate extracts were washed with water, brine, dried over anhydrous Na₂SO₄, and concentrated in vacuo. The residue was purified by column chromatography on silica gel eluting with a gradient of 0-20% of methanol/NH₄OH (99:1) in dichloromethane over 60 minutes to yield 1-[4-[([1,1′biphenyl]-2-ylcarbonyl)amino]benzoyl]-N-[2-(dimethylamino)ethyl]-1,2,3,4-tetrahydro-2′-(tert-butyldimethylsilyloxy)spiro[4H-1-benzazepine-4,1′-cylcopentane]-3′-carboxamide as a solid

Step J: 1-[4-[([1,1′biphenyl]-2-ylcarbonyl)amino]benzoyl]-N-[2-(dimethylamino)ethyl]-1,2,3,4-tetrahydro-2′-hydroxyspiro[4H-1-benzazepine-4,1′-cylcopentane]-3′-carboxamide

A solution of the compound prepared as in STEP I above (0.097 g, 0.13 mmol) in tetrahydrofuran (5 mL) was treated with 1 M tetrabutylammonium fluoride in tetrahydrofuran (0.26 mL, 0.26 mmol) and stirred for 2.5 hours at room temperature under a nitrogen atmosphere. The reaction mixture was partitioned between water and ethyl acetate and the ethyl acetate extract was washed with water, brine, dried over Na₂SO₄ and concentrated in vacuo to yield crude 1-[4-[([1,1′biphenyl]-2-ylcarbonyl)amino]benzoyl]-N-[2-(dimethylamino)ethyl]-1,2,3,4-tetrahydro-2′-hydroxyspiro[4H-1-benzazepine-4,1′-cylcopentane]-3′-carboxamide as a solid.

STEP K: 1-[4-[([1,1′-Biphenyl]-2-ylcarbonyl)amino]benzoyl]-N-[2-(dimethylamino)ethyl]-1,2,3,5-tetrahydro-2′-oxospiro[4H-1-benzazepine-4,1′-cyclopentane]-3′-carboxamide

The crude compound from STEP J (0.065 g, 0.103 mmol) was dissolved in dry dichloromethane (5 mL) and treated with the Dess-Martin periodinane (0.065 g, 0.15 mmol) while stirring at room temperature under an argon atmosphere. After 30 minutes, the reaction was quenched by the addition of 20% Na₂S₂O₃ (w/w) in saturated aqueous NaHCO₃ (5 mL total) and stirred for 15 minutes. The reaction mixture was extracted twice with ethyl acetate (15 mL) and the combined organic extracts were washed with saturated aqueous NaHCO₃, brine, dried over anhydrous Na₂SO₄ and concentrated in vacuo. The residue was purified via preparative thin layer chromatography on silica gel eluting with dichloromethane/methanol/NH₄OH (92:7.92:0.08) to yield the title compound as a white solid.

¹H NMR (300 MHz, CDCl₃) δ 1.45-1.65 (m, 4H), 2.15-2.60 (overlapping m, 6H), 2.30 (s, 6H), 2.78 (dd, 1H, J=12.7, 12.7 Hz), 3.25-3.70 (overlapping m, 3H), 5.13 (d, 1H, J=12.7 Hz); 6.63 (d, 1H, J=7.4 Hz), 6.90-7.70 (overlapping m, 16H)

MS (ES) m/z 629 (MH)⁺

### EXAMPLE 21

### 1-[4-[([1,1′-Biphenyl]-2-ylcarbonyl)amino]benzoyl]-N-[2-(dimethylamino)ethyl]-1,2,3,5-tetrahydro-2′-hydroxyspiro[4H-1-benzazepine-4,1′-cyclopentane]-3′-carboxamide

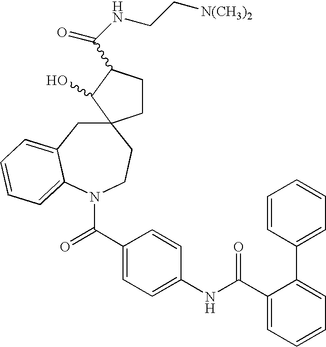

1-[4-[([1,1′-Biphenyl]-2-ylcarbonyl)amino]benzoyl]-N-[2-(dimethylamino)ethyl]-1,2,3,5-tetrahydro-2′-hydroxyspiro[4H-1-benzazepine-4,1′-cyclopentane]-3′-carboxamide was prepared according to the procedure described in Example 20 above, with substitution of suitably substituted reagents, to yield crude product, which was purified via preparative thin layer chromatography on silica gel eluting with dichloromethane/methanol/NH₄OH (92:7.92:0.08) to yield the title compound as a white solid.

¹H NMR (300 MHz, CDCl₃) δ 1.15-1.30 (m, 1H), 1.30-1.50 (m, 1H), 1.60-2.10 (m, 2H), 2.20-2.90 (overlapping m, 7H), 2.37 (s, 6H), 3.17 (d, 1H, J=13.2 Hz), 3.50-3.95 (overlapping m, 3H), 4.95 (d, 1H, J=12.7 Hz); 6.89 (d, 1H, J=7.3 Hz), 6.70-7.95 (overlapping m, 16H)

MS (ES) m/z 631 (MH)⁺

### EXAMPLE 22

### N-[4-[(1,2,3,5-Tetrahydro-2′-oxospiro[4H-1-benzazepine-4,1′-cyclopentan]-1-yl)carbonyl]phenyl]-[1,1′-biphenyl]-2-carboxamide

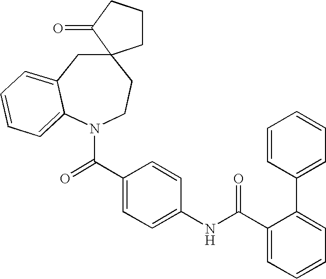

Step A: 1-(4-methylphenyl)sulfonyl)-1,2,3,4-tetrahydrospiro[4H-1-benzazepine-4,1′-cyclopen]-2′-one

The compound prepared as in STEP C of EXAMPLE 20 above, (6.70 g, 15.2 mmol) was combined with ethanol (23 mL), acetic acid (23 mL), and 6 N aqueous HCl (23 mL) and heated at reflux while stirring for 2.5 hours. The reaction mixture was cooled to room temperature and concentrated in vacuo to yield 1-(4-methyl phenyl)sulfonyl)-1,2,3,4-tetrahydrospiro[4H-1-benzazepine-4,1′-cyclopen]-2′-one as a solid.

Step B: 1,2,3,4-tetrahydrospiro[4H-1-benzazepine-4,1′-cyclopen]-2′-one

The compound prepared as in STEP A above (0.50 g, 1.35 mmol) was dissolved in anhydrous methanol (27 mL) and combined with magnesium turnings (0.656 g, 27 mmol) and heated at reflux while magnetically stirring under an argon atmosphere over 18 hours. The reaction was cooled to room temperature, filtered through filter agent, and concentrated in vacuo. The residue was triturated 3 times with ethyl acetate and the combined ethyl acetate triturations were filtered through filter agent. The filtrate was extracted with twice with brine, dried over anhydrous Na₂SO₄, and concentrated in vacuo to yield 1,2,3,4-tetrahydrospiro[4H-1-benzazepine-4,1′-cyclopen]-2′-one as a solid.

Step C: N-[4-[(1,2,3,5-Tetrahydro-2′-oxospiro[4H-1-benzazepine-4,1′-cyclopentan]-1-yl)carbonyl]phenyl]-[1,1′-bi phenyl]-2-carboxamide

To a slurry of 4-[([1,1′-biphenyl]-2-ylcarbonyl)amino]benzoic acid (0.444 g, 1.40 mmol; CAS 168626-74-2; WO 0132639) in dichloromethane (10 mL) was added N,N-dimethylformamide (0.1 mL) and thionyl chloride (0.204 mL, 2.80 mmol) while stirring at room temperature under an argon atmosphere. After 18 hours, the resulting solution was concentrated in vacuo, dissolved in dry toluene, concentrated in vacuo, and dissolved in 10 mL of dichloromethane. The resulting solution of acid chloride was added dropwise to a solution of the compound prepared in STEP B above (0.295 g, 1.12 mmol), and triethylamine (0.470 mL, 3.37 mmol) in dichloromethane (10 mL) while stirring at room temperature. After 24 hours, the reaction mixture was quenched with saturated aqueous NaHCO₃ and extracted with ethyl acetate. The ethyl acetate layer was extracted with saturated aqueous NaHCO₃, brine, dried over anhydrous Na₂SO₄, and concentrated in vacuo. The residue was purified via column chromatography on silica gel eluting with ethyl acetate/hexane (11:9) to yield the title compound as a white solid.

¹H NMR (300 MHz, DMSO-d₆) δ 1.40-1.70 (m, 2H), 1.70-2.00 (m, 2H), 2.20-2.50 (m, 2H), 2.59 (d, 1H, J=13.8 Hz), 2.72 (dd, 1H, J=13.1, 13.1 Hz), 2.97 (d, 1H, J=13.8 Hz), 4.77 (d, 1H, J=13.1 Hz); 6.69 (d, 1H, J=7.0 Hz), 6.90-7.70 (overlapping m, 16H)

MS (ES) m/z 515 (MH)⁺

### EXAMPLE 23

### N-[4-[(1,2,3,5-Tetrahydro-2′-hydroxyspiro[4H-1-benzazepine-4,1′-cyclopentan]-1-yl)carbonyl]phenyl]-[1,1′-biphenyl]-2-carboxamide

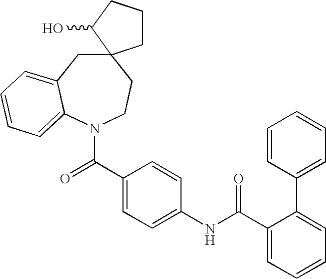

The compound prepared as in EXAMPLE 22 above (0.025 g, 0.048 mmol) was dissolved in methanol (1 mL) and treated with sodium borohydride (0.010 g, 0.288 mmol) while stirring at room temperature under an argon atmosphere. After 24 hours, the reaction mixture was concentrated in vacuo and the residue was partitioned between water and dichloromethane. The organic layer was separated, concentrated in vacuo, and purified via preparative thin layer chromatography on silica gel eluting with dichloromethane/methanol (97.5:2.5) to yield the title compound as a white solid.

¹H NMR (300 MHz, CD₃OD) δ 1.00-3.60 (overlapping m, 2H), 3.80-3.90 (m, 1H), 4.60-4.90 (m, 1H); 6.50-6.80 (m, 1H), 6.90-7.70 (overlapping m, 16H)

MS (ES) m/z 517 (MH)⁺

### EXAMPLE 24

### N-[3-Methoxy-4-[(1,2,3,5-tetrahydro-2′-oxospiro[4H-1-benzazepine-4,1′-cyclopentan]-1-yl)carbonyl]phenyl]-2-chloro-5-fluorobenzamide

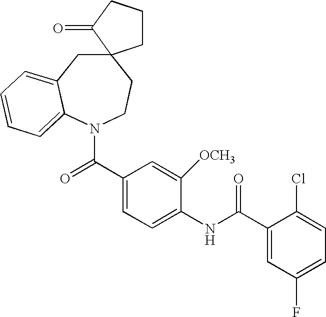

Step A: 4-(2-Chloro-5-fluoro-benzoylamino)-3-methoxy-benzoic acid

A slurry of 2-chloro-4-fluorobenzoic acid (3.15 g, 18.0 mmol) in thionyl chloride (15 mL) was stirred at room temperature under an argon atmosphere over 18 hours. The reaction mixture, which now appears as a solution, was concentrated in vacuo and dissolved in dichloromethane (10 mL). The resulting solution of acid chloride was added dropwise at 5° C. to a solution of methyl 3-methoxy-4-aminobenzoate (3.26 g, 18.0 mmol) and triethylamine (5.0 mL, 36 mmol) in dichloromethane (25 mL) while stirring under an argon atmosphere. The reaction mixture was allowed to slowly warm to room temperature over 18 hours. Water was added and the reaction mixture was stirred for 5 minutes. The layers were separated and the organic layer was extracted sequentially with 1 N aqueous HCl, saturated aqueous NaHCO₃, brine, dried over anhydrous MgSO₄, and concentrated in vacuo to yield methyl 4-(2-chloro-5-fluoro-benzoylamino)-3-methoxybenzoate. This material was suspended in tetrahydrofuran/water (1:1) (40 mL total), treated with lithium hydroxide monohydrate (0.755 g, 18.0 mmol) and stirred at room temperature over 18 hours. The reaction mixture was acidified to pH 2 with 1 M aqueous KHSO₄ and the resulting precipitate was isolated by filtration and recrystallized from a mixture of ethanol and ethyl acetate to yield 4-(2-chloro-5-fluoro-benzoylamino)-3-methoxy-benzoic acid as a white solid.

Step B: N-[3-Methoxy-4-[(1,2,3,5-tetrahydro-2′-oxospiro[4H-1-benzazepine-4,1′-cyclopentan]-1-yl)carbonyl]phenyl]-2-chloro-5-fluorobenzamide

A slurry of 4-(2-chloro-5-fluoro-benzoylamino)-3-methoxy-benzoic acid, prepared as in STEP A above (1.07 g, 3.31 mmol) in dichloromethane (25 mL) was treated with thionyl chloride (25 mL) and heated at reflux under an atmosphere of argon over 18 hours. The reaction mixture was concentrated in vacuo and dissolved in dichloromethane (50 mL). The resulting solution of acid chloride was added dropwise at 5° C. to a solution of the compound prepared as in STEP B of EXAMPLE 22 (0.640 g, 2.97 mmol), triethylamine (2 mL), and N,N-dimethylformamide (0.25 mL) in dichloromethane (25 mL) while stirring under an argon atmosphere. After 18 hours, the reaction mixture was extracted with saturated aqueous NaHCO₃. The organic layer was extracted with aqueous 1 M KHSO₄, brine, dried over anhydrous MgSO₄, and concentrated in vacuo. The residue was purified via column chromatography on silica gel eluting with a gradient of 0-5% methanol in dichloromethane over 60 minutes to yield the title compound as a white solid.

¹H NMR (300 MHz, CDCl₃) δ 1.50-1.65 (m, 2H), 1.65-1.80 (m, 2H), 1.80-2.00 (m, 2H), 2.04 (dd, 1H, J=13.8 Hz), 2.80 (dd, 1H, J=12.4, 12.4 Hz), 3.30 (d, 1H, J=13.9 Hz), 3.70 (s, 3H), 5.03 (d, 1H, J=13.8 Hz), 6.60-7.30 (overlapping m, 7H), 7.70-7.85 (m, 1H), 8.26 (d, 1H, J=8.3 Hz), 8.60 (s, 1H)

MS (ES) m/z 521 (MH)⁺.

### EXAMPLE 25

### 4-(3-ethoxycarbonyl-propyl)-1-(toluene-4-sulfonyl)-2,3,4,5-tetrahydro-1H-benzo[b]azepine-4-carboxylic acid ethyl ester

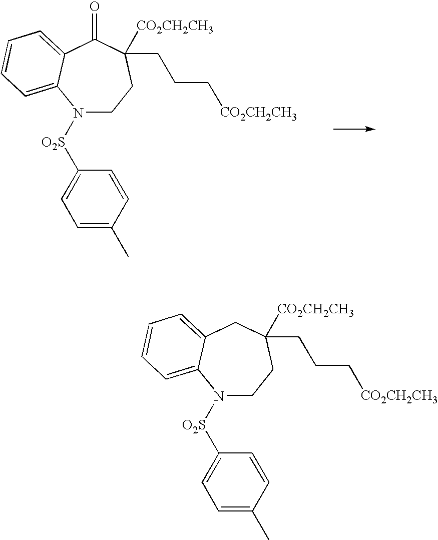

A series of experiments were run on the conversion of 4-(3-ethoxycarbonyl-propyl)-5-oxo-1-(toluene-4-sulfonyl)-2,3,4,5-tetrahydro-1H-benzo[b]azepine-4-carboxylic acid ethyl ester to 4-(3-ethoxycarbonyl-propyl)-1-(toluene-4-sulfonyl)-2,3,4,5-tetrahydro-1H-benzo[b]azepine-4-carboxylic acid ethyl ester.

The experiments were carried out in an automated reactor system using 12 reactors. The experiments were completed according the general procedure described below. The experimental conditions were selected based on a quadratic algorithmic design. The reagents, including starting materials were prepared by diluting with solvent to the concentration listed in Table 1.

TABLE 1

| Reagent | Concentration |
|---|---|
| Methane sulfonic acid | 1.83 mmol/mL |
| Trifluoroacetic acid | 0.93 mmol/mL |
| Boron trifluoride | 0.50 mmol/mL |
| etherate | |
| Triethylsilane | 1.55 mmol/mL |

Portions of 4-(3-ethoxycarbonyl-propyl)-5-oxo-1-(toluene-4-sulfonyl)-2,3,4,5-tetrahydro-1H-benzo[b]azepine-4-carboxylic acid ethyl ester (SM) were dissolved in dichloromethane and trifluoromethylbenzene to produce a solution containing 50 mg of SM/mL (0.1 mmol/mL) of solution. 2 mL of the appropriate solution (0.2 mmol of SM) was placed in the individual reactors and stirred for 5 minutes at 0° C. The selected amounts of trifluoroacetic acid, boron trifluoride etherate, and methanesulfonic acid were added to each reaction. The reaction was treated with the selected amount of triethylsilane. The reaction was then warmed to 23° C., held at that temperature for 30 minutes, and sampled. The temperature was held at 23° C. for an additional 30 minutes, then cooled to 0° C. The temperature was maintained at 0° C. until all samples could be manually removed from the equipment.

The conditions for each experiment (including equivalents of CH₃SO₃H, trifluoroacetic acid (TFA), BF₃.etherate and triethylsilane and the selected solvent used) were as listed in Table 2 below.

TABLE 2
Experimental Conditions

| Trial | Eq. | Eq. | Eq. | Eq. | |
|---|---|---|---|---|---|
| # | CH₃SO₃H | TFA | BF₃.Et₂O | Et₃SiH | Solvent |
| 21 | 5.28 | 4.65 | 1.88 | 8.0 | Trifluoromethylbenzene |
| 20 | 5.28 | 0.93 | 0.47 | 5.0 | Dichloroethane |
| 17 | 1.76 | 2.79 | 1.88 | 8.0 | Trifluoromethylbenzene |
| 16 | 3.52 | 0.93 | 1.88 | 2.0 | Trifluoromethylbenzene |
| 2 | 5.28 | 4.65 | 0.47 | 2.0 | Trifluoromethylbenzene |
| 1 | 1.76 | 0.93 | 0.47 | 2.0 | Dichloroethane |
| 6 | 1.76 | 4.65 | 1.88 | 8.0 | Dichloroethane |
| 4 | 1.76 | 4.65 | 1.88 | 2.0 | Trifluoromethylbenzene |
| 13 | 1.76 | 4.65 | 0.47 | 5.0 | Dichloroethane |
| 18 | 3.52 | 4.65 | 0.47 | 2.0 | Dichloroethane |
| 3 | 5.28 | 0.93 | 1.88 | 2.0 | Dichloroethane |
| 14 | 3.52 | 2.79 | 1.175 | 2.0 | Dichloroethane |
| 2 | 5.28 | 4.65 | 0.47 | 2.0 | Trifluoromethylbenzene |
| 12 | 1.76 | 0.93 | 1.88 | 5.0 | Dichloroethane |
| 8 | 5.28 | 0.93 | 1.88 | 8.0 | Dichloroethane |
| 15 | 1.76 | 4.65 | 1.175 | 8.0 | Trifluoromethylbenzene |
| 10 | 3.52 | 0.93 | 0.47 | 5.0 | Trifluoromethylbenzene |
| 9 | 1.76 | 2.79 | 0.47 | 5.0 | Trifluoromethylbenzene |
| 3 | 5.28 | 0.93 | 1.88 | 2.0 | Dichloroethane |
| 7 | 1.76 | 0.93 | 0.47 | 8.0 | Dichloroethane |
| 11 | 5.28 | 4.65 | 1.88 | 5.0 | Dichloroethane |
| 1 | 1.76 | 0.93 | 0.47 | 2.0 | Dichloroethane |
| 5 | 5.28 | 4.65 | 0.47 | 8.0 | Dichloroethane |
| 19 | 1.76 | 0.93 | 1.175 | 5.0 | Trifluoromethylbenzene |

The study as listed in Table 2 was completed in two parts. In Table 3 the distinction between the two runs is made by using an “A” after the reactor number.

The individual experiments were sampled and transferred for analysis by chromatography to determine % yield, with results as listed in Table 3. The analysis of all sample was performed on a Super ODS column using 0.1% trifluoroacetic acid in water/0.1% trifluoroacetic acid in acetonitrile gradient from 55% acetonitrile to 65% acetonitrile over 10 minutes.

TABLE 3

| Reactor | Trial # | % Yield | Reactor | Trial # | % Yield |
|---|---|---|---|---|---|
| 1 | 21 | 72 | 1A | 2 | 1 |
| 2 | 20 | 86 | 2A | 12 | 24 |
| 3 | 17 | 29 | 3A | 8 | 87 |
| 4 | 16 | 15 | 4A | 15 | 1 |
| 5 | 2 | 36 | 5A | 10 | 0 |
| 6 | 1 | 8 | 6A | 9 | 0 |
| 7 | 6 | 88 | 7A | 3 | 55 |
| 8 | 4 | 16 | 8A | 7 | 1 |
| 9 | 13 | 13 | 9A | 11 | 61 |
| 10 | 18 | 29 | 10A | 1 | 0 |
| 11 | 3 | 80 | 11A | 5 | 39 |
| 12 | 14 | 66 | 12A | 19 | 0 |

### EXAMPLE 26

### (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid diethylamine salt

A solution of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid (1.65 g, 3.01 mmol) and diethylamine (0.22 g, 3.01 mmol) in 20 mL of isopropyl acetate and 1 mL of methanol was heated to reflux to distill off methanol. Water (0.108 g, 6.02 mmol) was then added. The clear solution was cooled to 60° C. and solid salt precipitated. The resulting solution was then cooled to ambient temperature, and the solid was collected by filtration and dried in a vacuum oven at 55° C.

¹H NMR of the isolated salt indicated that the salt was a one to one molar ratio of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid and diethylamine without any residual solvents.

¹H NMR (DMSO-d₆) δ9.76 (s, 1H), 7.85 (d, J=8.5 Hz, 1H), 7.60 to 6.65 (m, 9H), 6.37 and 5.93 (ss, 1H, rotamers), 4.82-4.58 (m, 1H), 3.57 (s, 3H), 3.35. (t, J=13.3 Hz, 1H), 3.03 to 2.78 (m, 1H), 2.67 (q, J=7.2 Hz, 4H), 2.75 to 2.33 (m, 3H), 2.08 to 1.80 (m, 2H), 1.73 to 1.33 (m, 2H), 1.07 (t, J=7.2 Hz, 6H).

mp: 159.7-160.4° C.

Solubility in water: >10 mg/mL.

Chemical Analysis for C₃₄H₃₇ClFN₃O₅.0.2H₂O: Calc'd: C, 65.27; H, 6.03; N, 6.72; F, 3.04; Cl, 5.67; KF, 0.58. Found: C, 65.29; H, 5.69; N, 6.62; F, 3.12; Cl, 5.76; KF, 0.53.

### EXAMPLE 27

### (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid piperazine salt

A solution of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid (2.78 g, 5.06 mmol) and piperazine (0.22 g, 2.53 mmol) in 30 mL of isopropyl acetate and 6 mL of methanol was heated to reflux to distill off methanol. Water (0.091 g, 5.06 mmol) was then added. The clear solution was cooled to 72° C. and the solid salt precipitated. The resulting solution was cooled to ambient temperature, and the solid collected by filtration and dried in a vacuum oven at 55° C.

¹H NMR of the isolated salt indicated that the salt was in two to one molar ratio of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid and piperazine with 0.36 mole equivalent of isopropyl acetate.

¹H NMR (DMSO-d⁶) δ9.76 (s, 1H), 7.85 (d, J=8.5 Hz, 1H), 7.61 to 6.68 (m, 9H), 6.52 and 6.02 (ss, 1H, rotamers), 4.82-4.58 (m, 1H), 3.57 (s, 3H), 3.35 (t, J=13.3 Hz, 1H), 3.03 to 2.33 (m, 4H), 2.73 (s, 4H), 2.08 to 1.80 (m, 2H), 1.73 to 1.33 (m, 2H).

mp: 159-161° C.

Chemical Analysis for C₃₂H₃₁ClFN₃O₅.0.36C₅H₁₀O₂.0.45H₂O Calc'd: C, 63.74; H, 5.62; N, 6.60; F, 2.99; Cl, 5.57; KF, 1.28. Found: C, 63.60, H, 5.35, N, 6.51, F, 3.12, Cl, 5.77; KF, 1.28.

### EXAMPLE 28

### (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid 1-(2-hydroxyethyl)pyrrolidine salt

A solution of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid (2.48 g, 4.51 mmol) and 1-(2-hydroxyethyl)pyrrolidine (1.04 g, 9.02 mmol) in 46 mL of isopropyl acetate and 2 mL of methanol was heated to reflux to distill off methanol. Water (0.086 g, 4.51 mmol) was than added. The solid salt started to precipitate upon cooling. The solution was cooled to the ambient temperature, and the solid (2.47 g) was collected by filtration.

¹H NMR of the isolated salt indicated that the salt was in one to one molar ratio of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid and 1-(2-hydroxyethyl)pyrrolidine without any residual solvents.

¹H NMR (CD₃OD) δ8.04 (d, J=8.3 Hz, 1H), 7.57 to 6.67 (m, 9H), 6.44 and 6.12 (ss, 1H, rotamers), 3.81 (t, J=5.3 Hz, 2H), 3.63 (s, 3H), 3.45 to 2.94 (m, 3H), 3.28 (m, 4H), 3.20 (t, J=5.3 Hz, 2H), 2.85 to 2.48 (m, 3H), 2.18 to 1.89 (m, 2H), 2.04 (m, 4H), 1.83 to 1.45 (m, 2H).

mp: 187.5-188.4° C.

Solubility in water: >20 mg/mL.

Chemical Analysis for C₃₀H₂₆ClFN₂O₅.C₆H₁₃NO.H₂O Calc'd: C, 63.38; H, 6.06; N, 6.16; Cl, 5.20; F, 2.78. Found: C, 63.32; H, 5.57; N, 5.95; Cl, 5.25; F, 3.17.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A process for the preparation of a compound of formula (I) 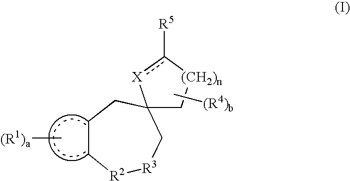
wherein 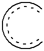
is selected from the group consisting aryl and heteroaryl; provided that the heteroaryl group does not contain a nitrogen atom;
a is an integer from 1 to 3;
R¹ is selected from the group consisting of hydrogen, halogen, hydroxy, alkoxy, phenyl, substituted phenyl, alkylthio, arylthio, alkyl-sulfoxide, aryl-sulfoxide, alkyl-sulfone and aryl-sulfone;
—R²—R³— is selected from the group consisting of 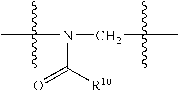
and 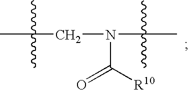
R¹⁰ is selected from the group consisting of alkyl, substituted alkyl, phenyl, substituted phenyl, heteroaryl, substituted heteroaryl and —(B)₀₋₁-G-(E)₀₋₁-(W)₁₋₃;
wherein B is selected from (CH₂)₁₋₃, NH or O;
G is selected from aryl, substituted aryl, heteroaryl or substituted heteroaryl;
E is selected from —O—, —S—, —NH—, —(CH₂)₀₋₃—N(R¹¹)C(O)— or —(CH₂)₀₋₃—C(O)NR¹¹—; wherein R¹¹ is selected from the group consisting of hydrogen, alkyl an substituted alkyl;
each W is independently selected from hydrogen, alkyl, substituted alkyl, amino, substituted amino, alkylthiophenyl, alkyl-sulfoxidephenyl, aryl, substituted aryl, heteroaryl or substituted heteroaryl;
X is selected from the group consisting of CH, CH₂, CHOH and C(O); [symbol]
represents a single or double bond;
provided that when R¹ is iodine, bromine, alkylthio, arylthio, alkyl-sulfone or aryl-sulfone, then [symbol] is a double bond;
n is an integer from 1 to 3;
b is an integer from 1 to 2;
R⁴ is selected from the group consisting of hydrogen, alkyl, substituted alkyl, phenyl and substituted phenyl;
R⁵ is selected from the group consisting of hydrogen, alkyl, substituted alkyl, aldehyde, carboxyl, alkoxycarbonyl, substituted alkoxycarbonyl, —(CH₂)ₖNZ¹Z² and —C(O)NZ¹Z²;
wherein k is an integer from 1 to 4;
Z¹ and Z² are independently selected from hydrogen, alkyl, substituted alkyl, heterocyclyl, substituted heterocyclyl, aminocarbonyl or substituted aminocarbonyl;
alternatively Z¹ and Z² are taken together with the N atom to which they are bound to form a heterocyclyl, substituted heterocyclyl, heteroaryl or substituted heteroaryl;
or an optical isomer, enantiomer, diastereomer, racemate thereof, or a pharmaceutically acceptable salt thereof;
comprising 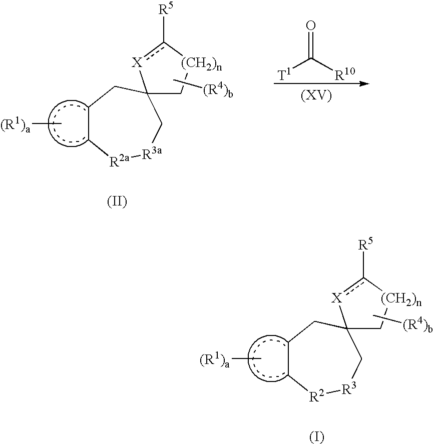
reacting a compound of formula (II) wherein —R^{2a}—R^{3a}— is selected from the group consisting of —NH—CH₂— and —CH₂—NH— with a compound of formula (XV) wherein T¹ is Cl, Br or F; in the presence of a base capable of neutralizing HT¹; in a non-alcoholic organic solvent or a mixture of a non-alcoholic organic solvent and water, to yield the corresponding compound of formula (I).

2. The process as in claim 1 wherein T¹ is Cl.

3. The process as in claim 1 wherein R⁵ in the compound of formula (II) is carboxyl, further comprising protecting the carboxyl by reacting the compound of formula (II) with TMSCl in situ.

4. The process as in claim 1 wherein 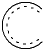
is phenyl, X is —CH₂—, R⁵ is —CO₂H, n is 1, b is 0, —R²—R³— is 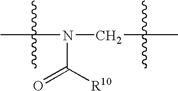
and R¹⁰ is

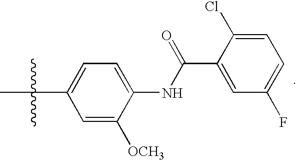

5. The process as in claim 4 wherein the base capable of neutralizing HT¹ is an organic tertiary amine base.

6. The process as in claim 5 wherein the compound of formula (II) is reacted with the compound of formula (XV) in a non-alcoholic organic solvent.

7. The process as in claim 6 wherein the organic tertiary amine base is pyridine and the non-alcoholic organic solvent is toluene.

8. The process as in claim 7 wherein the compound of formula (II) is reacted with the compound of formula (XV) at a temperature in the range of between about 0° C. and about room temperature.

9. A compound prepared according to the process of claim 1.

10. A compound prepared according to the process of claim 4.

11. A process for the preparation of a compound of formula (II) 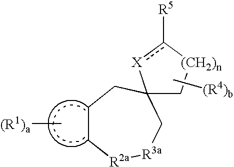
wherein 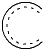
is selected from the group consisting aryl and heteroaryl; provided that the heteroaryl does not contain a nitrogen atom;
a is an integer from 1 to 3;
R¹ is selected from the group consisting of hydrogen, halogen, hydroxy, alkoxy, phenyl, substituted phenyl, alkylthio, arylthio, alkyl-sulfoxide, aryl-sulfoxide, alkyl-sulfone and aryl-sulfone;
—R^{2a}—R^{3a}— is selected from the group consisting of —NH—CH₂— and —CH₂—NH—;
X is selected from the group consisting of CH, CH₂, CHOH and C(O); [symbol]
represents a single or double bond;
provided that [symbol] when R¹ is iodine, bromine, alkylthio, arylthio, alkyl-sulfone or aryl-sulfone, then is a double bond;
n is an integer from 1 to 3;
b is an integer from 1 to 2;
R⁴ is selected from the group consisting of hydrogen, alkyl, substituted alkyl, phenyl and substituted phenyl;
R⁵ is selected from the group consisting of hydrogen, alkyl, substituted alkyl, aldehyde, carboxyl, alkoxycarbonyl, substituted alkoxycarbonyl, —(CH₂)ₖNZ¹Z² and —C(O)NZ¹Z²;
wherein k is an integer from 1 to 4;
Z¹ and Z² are independently selected from hydrogen, alkyl, substituted alkyl, heterocyclyl, substituted heterocyclyl, aminocarbonyl or substituted aminocarbonyl;
alternatively Z¹ and Z² are taken together with the N atom to which they are bound to form a heterocyclyl, substituted heterocyclyl, heteroaryl or substituted heteroaryl;
or an optical isomer, enantiomer, diastereomer, racemate thereof, or a pharmaceutically acceptable salt thereof;
comprising 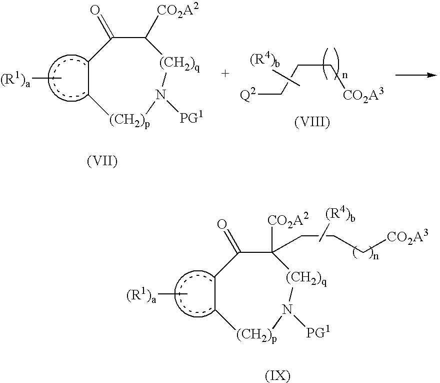
reacting a compound of formula (VII), wherein p is an integer from 0 to 1, q is an integer from 1 to 2, provided that when p is 0 then q is 2 and when p is 1 then q is 1, PG¹ is a nitrogen protecting group and A² is lower alkyl; with a compound of formula (VIII) wherein Q² is a leaving group and A³ is lower alkyl; in the presence of a base capable of deprotonating an alpha proton to the ketone on the compound of formula (VII); in an aprotic solvent, to yield the corresponding compound of formula (IX); 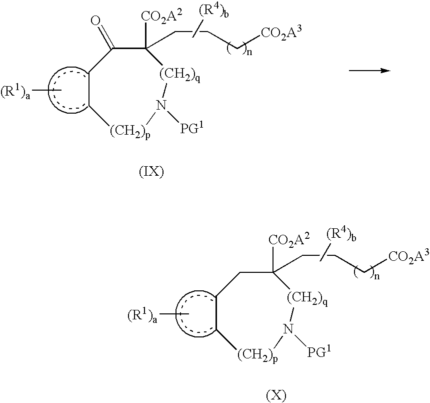
reducing the compound of formula (IX) to yield the corresponding compound of formula (X); 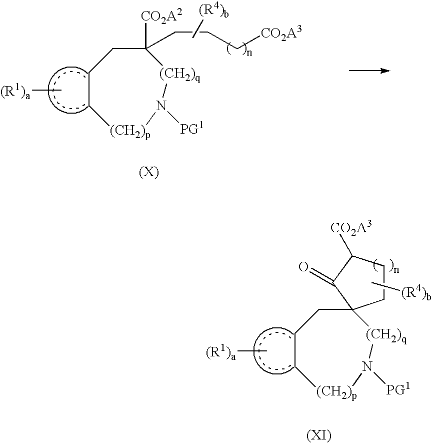
reacting the compound of formula (X) in the presence of a base capable of deprotonating an alpha proton to the CO₂A³ substituent; in an organic solvent that does not prevent the deprotonation of an alpha proton to the CO₂A³ substituent, to yield the corresponding compound of formula (XI); 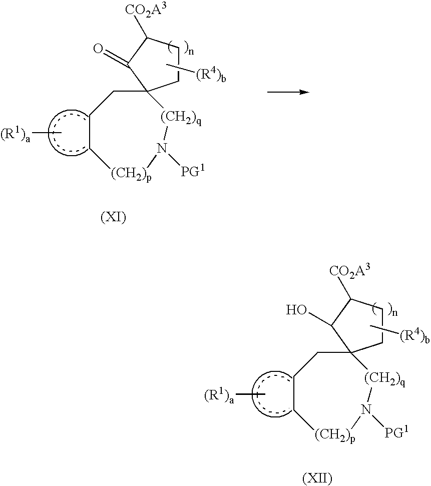
reducing the compound of formula (XI), to yield the corresponding compound of formula (XII); and 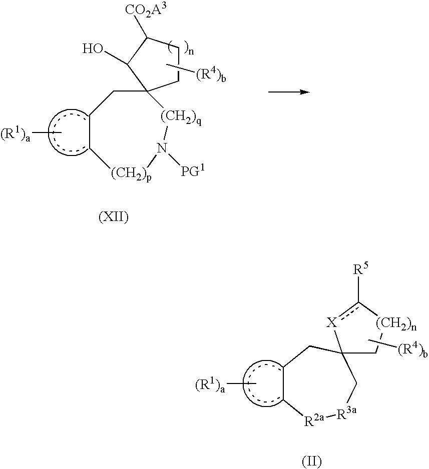
reacting the compound of formula (XII), to yield the corresponding compound of formula (II).

12. The process as in claim 11 wherein 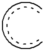
is phenyl, X is —CH₂—, R⁵ is —CO₂H, n is 1, b is 0 and —R^{2a}—R^{3a}— is —NH—CH₂—.

13. The process as in claim 12, wherein the base capable of deprotonating an alpha proton to the ketone on the compound of formula (VII) is an inorganic base.

14. The process as in claim 13, wherein the inorganic base is K₂CO₃ and the aprotic solvent in DMF.

15. The process as in claim 12, wherein the compound of formula (IX) is reduced to the corresponding compound of formula (X) by reacting the compound of formula (IX) with triethylsilane in the presence of a mixture of TFA, methanesulfonic acid and BF₃.etherate.

16. The process as in claim 15, wherein the triethylsilane, TFA, methanesulfonic acid and BF₃.etherate are present in a molar equivalent ratio of 5.0 triethylsilane to 2.5 TFA to 6.0 metahnesulfonic acid to 1.8 BF₃.etherate.

17. The process as in claim 16, wherein the compound of formula (IX) is reduced to the corresponding compound of formula (X) in dichloroethane.

18. The process as in claim 12, wherein the base capable of deprotonating an alpha proton to the CO₂A³ substituent is an alkali metal alkoxide.

19. The process as in claim 18, wherein the alkali metal alkoxide is potassium t-butoxide and wherein the organic solvent that does not prevent the deprotonation of an alpha proton to the CO₂A³ substituent is toluene.

20. The process as in claim 12, wherein the compound of formula (XI) is reduced to the corresponding compound of formula (XII) by reacting the compound of formula (XI) with sodium borohydride in methanol.

21. A compound prepared according to the process of claim 11.

22. A compound prepared according to the process of claim 12.

23. A process for the preparation of a compound of formula (XVa) 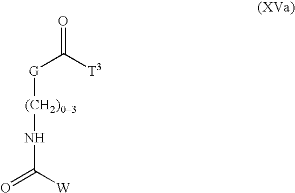
wherein
T³ is selected from the group consisting of Cl, Br and F;
G is selected from aryl, substituted aryl, heteroaryl or substituted heteroaryl;
W is selected from hydrogen, alkyl, substituted alkyl, amino, substituted amino, alkylthiophenyl, alkyl-sulfoxidephenyl, aryl, substituted aryl, heteroaryl or substituted heteroaryl;
comprising 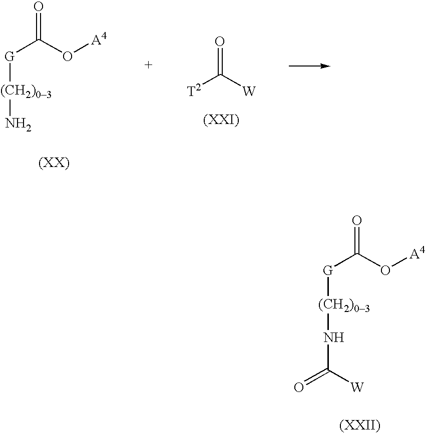
reacting a compound of formula (XX), wherein A⁴ is lower alkyl with a compound of formula (XXI) wherein T² is Cl, Br or F; in the presence of a base capable of neutralizing HT²; in a non-alcoholic organic solvent, to yield the corresponding compound of formula (XXII); 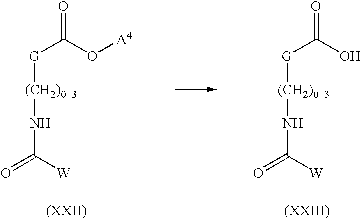
hydrolyzing the compound of formula (XXII), to yield the corresponding compound of formula (XXIII); 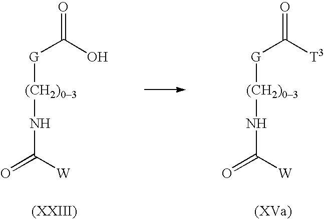
reacting the compound of formula (XXIII) with a reagent capable of converting the —CO₂H substituent to the corresponding —C(O)T³ substituent; in an inert organic solvent, to yield the corresponding compound of formula (XVa).

24. The process as in claim 23, wherein T³ is Cl.

25. The process as in claim 24, wherein G is 1-(3-methoxy-phenyl) and W is 1-(2-chloro-5-fluoro-phenyl).

26. The process as in claim 25, wherein the base capable of neutralizing HT² is an organic tertiary amine base.

27. The process as in claim 26, wherein the organic tertiary amine base is triethylamine.

28. The process as in claim 27, wherein the non-alcoholic organic solvent is DCM or ethyl acetate.

29. The process as in claim 28, wherein the compound of formula (XX) is reacted with the compound of formula (XXI) at a temperature in the range of between about 0° C. and about room temperature.

30. The process as in claim 25, wherein the compound of formula (XXII) is hydrolyzed to the corresponding compound of formula (XXIII) by reacting the compound of formula (XXII) with water in the presence of base, in an organic solvent.

31. The process as in claim 30, wherein the base is LiOH and the organic solvent is THF.

32. The process as in claim 25, wherein the reagent capable of converting the —CO₂H substituent to the corresponding —C(O)T³ substituent is oxalyl chloride or thionyl chloride.

33. The process as in claim 32, wherein the compound of formula (XXIII) is reacted with oxalyl chloride at a temperature in the range of between about 0° C. and about room temperature.

34. A compound prepared according to the process of claim 23.

35. A compound prepared according to the process of claim 25.

36. A compound of formula (II) 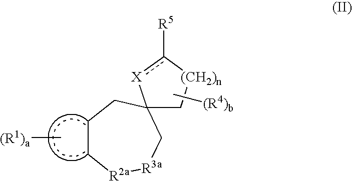 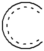
is selected from the group consisting aryl and heteroaryl; provided that the heteroaryl does not contain a nitrogen atom;
a is an integer from 1 to 3;
R¹ is selected from the group consisting of hydrogen, halogen, hydroxy, alkoxy, phenyl, substituted phenyl, alkylthio, arylthio, alkyl-sulfoxide, aryl-sulfoxide, alkyl-sulfone and aryl-sulfone;
—R^{2a}—R^{3a}— is selected from the group consisting of —NH—CH₂— and —CH₂—NH—;
X is selected from the group consisting of CH, CH₂, CHOH and C(O); [symbol]
represents a single or double bond;
provided that when R¹ is iodine, bromine, alkylthio, arylthio, alkyl-sulfone or aryl-sulfone, then [symbol] is a double bond;
n is an integer from 1 to 3;
b is an integer from 1 to 2;
R⁴ is selected from the group consisting of hydrogen, alkyl, substituted alkyl, phenyl and substituted phenyl;
R⁵ is selected from the group consisting of hydrogen, alkyl, substituted alkyl, aldehyde, carboxyl, alkoxycarbonyl, substituted alkoxycarbonyl, —(CH₂)ₖNZ¹Z² and —C(O)NZ¹Z²;
wherein k is an integer from 1 to 4;
Z¹ and Z² are independently selected from hydrogen, alkyl, substituted alkyl, heterocyclyl, substituted heterocyclyl, aminocarbonyl or substituted aminocarbonyl;
alternatively Z¹ and Z² are taken together with the N atom to which they are bound to form a heterocyclyl, substituted heterocyclyl, heteroaryl or substituted heteroaryl;
or an optical isomer, enantiomer, diastereomer, racemate thereof, or a pharmaceutically acceptable salt thereof.

37. A compound as in claim 36, wherein 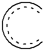
is phenyl, X is —CH₂—, R⁵ is —CO₂H, n is 1, b is 0 and —R^{2a}—R^{3a}— is —NH—CH₂—.

38. A compound as in claim 36 selected from
1,2,3,5-tetrahydro-spiro[4H-1-benzazepine-4,1′-[2]cyclopentene]-3′-carboxylic acid;
or a pharmaceutically acceptable salt thereof.

39. A compound as in claim 36 selected from
(4R)-1,2,3,5-tetrahydro-spiro[4H-1-benzazepine-4,1′-[2]cyclopentene]-3′-carboxylic acid;
or a pharmaceutically acceptable salt thereof.

40 A compound as in claim 36 selected from
(4S)-1,2,3,5-tetrahydro-spiro[4H-1-benzazepine-4,1′-[2]cyclopentene]-3′-carboxylic acid;
or a pharmaceutically acceptable salt thereof.

41. A process for the preparation of (4R)-1,2,3,5-tetrahydro-spiro[4H-1-benzazepine-4,1′-[2]cyclopentene]-3′-carboxylic acid comprising reacting a racemic mixture of 1,2,3,5-tetrahydro-spiro[4H-1-benzazepine-4,1′-[2]cyclopentene]-3′-carboxylic acid with (−)-camphorsulfonic acid.

42. The process as in claim 41, wherein the (−)-camphorsulfonic acid is present in an amount equal to about one equivalent.

43. The process as in claim 42, wherein the 1,2,3,5-tetrahydro-spiro[4H-1-benzazepine-4,1′-[2]cyclopentene]-3′-carboxylic acid is reacted with the (−)-camphorsulfonic acid in methanol.

44. A process for the preparation of (4S)-1,2,3,5-tetrahydro-spiro[4H-1-benzazepine-4,1′-[2]cyclopentene]-3′-carboxylic acid comprising reacting a racemic mixture of 1,2,3,5-tetrahydro-spiro[4H-1-benzazepine-4,1′-[2]cyclopentene]-3′-carboxylic acid with (+)-camphorsulfonic acid.

45. A compound of the formula

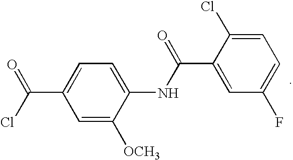

46. A pharmaceutical composition comprising a pharmaceutically acceptable carrier and a compound of claim 9.

47. A pharmaceutical composition made by mixing a compound of claim 9 and a pharmaceutically acceptable carrier.

48. A process for making a pharmaceutical composition comprising mixing a compound of claim 9 and a pharmaceutically acceptable carrier.

49. A method of treating a conditions involving increased vascular resistance and cardiac insufficiency, in a subject in need thereof comprising administering to the subject a therapeutically effective amount of the compound of claim 9.

50. The method of claim 49, wherein the condition is selected from the group consisting of aggression, obsessive-compulsive disorders, hypertension, dysmenorrhea, congestive heart failure/cardiac insufficiency, coronary vasospasm, cardiac ischemia, liver cirrhosis, renal vasospasm, renal failure, edema, ischemia, stroke, thrombosis, water retention, nephritic syndrome and central nervous system injuries.

51. The process according to claim 15, wherein the triethylsilane, BF₃.Etherate, TFA, and methanesulfonic acid are present in a molar ratio of 3.75 triethylsilane to 2.79 BF₃-Etherate to 5.27 TFA to 1.2 methanesulfonic acid.

52. A diethylamine salt of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, said acid being compound of formula (Ia).

53. A diethylamine salt as in claim 52 wherein the ratio of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid to diethylamine is 1:1.

54. A diethylamine salt as in claim 53 comprising the following X-ray diffraction peaks:

| Position [°2θ] | d-spacing [Å] | Relative Intensity [%] |
|---|---|---|
| 12.4469 | 7.1116 | 13.10 |
| 13.6758 | 6.4751 | 15.99 |
| 13.9948 | 6.3283 | 45.16 |
| 16.0254 | 5.5307 | 29.23 |
| 16.4868 | 5.3769 | 15.27 |
| 17.1962 | 5.1567 | 60.20 |
| 17.6157 | 5.0348 | 18.08 |
| 19.2580 | 4.6090 | 10.28 |
| 20.2682 | 4.3815 | 78.24 |
| 20.7710 | 4.2766 | 19.85 |
| 21.1852 | 4.1939 | 50.33 |
| 22.4210 | 3.9654 | 14.46 |
| 23.1866 | 3.8330 | 44.46 |
| 23.2845 | 3.8203 | 50.44 |
| 23.7616 | 3.7447 | 44.86 |
| 24.1721 | 3.6820 | 38.54 |
| 24.5539 | 3.6256 | 13.46 |
| 25.4790 | 3.4960 | 20.14 |
| 26.4543 | 3.3693 | 100.00 |
| 27.2074 | 3.2777 | 47.26 |
| 27.6733 | 3.2236 | 24.27 |
| 29.3616 | 3.0420 | 15.82 |
| 31.9613 | 2.7979 | 10.81 |
| 32.3129 | 2.7683 | 11.11 |

55. A piperazine salt of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, said acid being compound of formula (Ia).

56. A piperazine salt as in claim 55 wherein the ratio of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid to piperazine is 2:1.

57. A piperazine salt as in claim 56 comprising the following X-ray diffraction peaks:

| Position [°2θ] | d-spacing [Å] | Relative Intensity [%] |
|---|---|---|
| 13.5395 | 6.5400 | 21.78 |
| 14.8734 | 5.9564 | 25.04 |
| 15.4039 | 5.7524 | 12.84 |
| 15.8609 | 5.5877 | 52.16 |
| 16.5948 | 5.3421 | 15.00 |
| 18.5405 | 4.7857 | 31.72 |
| 19.1470 | 4.6355 | 92.10 |
| 19.6968 | 4.5073 | 55.49 |
| 20.1348 | 4.4102 | 68.92 |
| 20.7233 | 4.2863 | 12.82 |
| 21.3009 | 4.1714 | 10.41 |
| 22.1553 | 4.0124 | 17.97 |
| 22.8971 | 3.8841 | 29.11 |
| 23.9903 | 3.7095 | 24.43 |
| 24.7962 | 3.5907 | 27.08 |
| 25.8556 | 3.4460 | 100.00 |
| 26.5390 | 3.3587 | 40.92 |
| 27.1754 | 3.2815 | 41.38 |
| 27.5201 | 3.2412 | 40.85 |
| 28.9219 | 3.0872 | 26.04 |
| 30.0687 | 2.9720 | 14.28 |
| 31.6571 | 2.8264 | 10.40 |
| 26.4543 | 3.3693 | 100.00 |
| 33.5897 | 2.6681 | 10.85 |

58. A 1-(2-hydroxyethyl)pyrrolidine salt of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid, said acid being compound of formula (Ia).

59. A 1-(2-hydroxyethyl)pyrrolidine salt as in claim 58 wherein the ratio of (4R)-1-[4-(2-chloro-5-fluorobenzoyl)amino-3-methoxybenzoyl]-1,2,3,5-tetrahydro-spiro[4H-1-benzazepine-4,1′-[2]cyclopentene]-3′-carboxylic acid to 1-(2-hydroxyethyl)pyrrolidine is 1:1.

60. A 1-(2-hydroxyethyl)pyrrolidine salt as in claim 59 comprising the following X-ray diffraction peaks:

| Position [°2θ] | d-spacing [Å] | Relative Intensity [%] |
|---|---|---|
| 12.4052 | 7.1353 | 35.63 |
| 14.5331 | 6.0950 | 27.39 |
| 15.8254 | 5.6001 | 100.00 |
| 16.1407 | 5.4914 | 25.15 |
| 17.0466 | 5.2016 | 10.01 |
| 17.5261 | 5.0604 | 36.71 |
| 18.8205 | 4.7151 | 33.63 |
| 19.3437 | 4.5888 | 10.85 |
| 19.6767 | 4.5119 | 16.22 |
| 20.0173 | 4.4358 | 17.78 |
| 20.4608 | 4.3407 | 29.62 |
| 20.6769 | 4.2958 | 23.59 |
| 21.7248 | 4.0909 | 16.51 |
| 22.1398 | 4.0152 | 21.99 |
| 22.6780 | 3.9211 | 86.85 |
| 23.3486 | 3.8100 | 56.43 |
| 23.9247 | 3.7195 | 75.49 |
| 24.4967 | 3.6339 | 36.16 |
| 25.0891 | 3.5495 | 24.11 |
| 25.3622 | 3.5119 | 36.04 |
| 27.6456 | 3.2268 | 17.69 |
| 29.1634 | 3.0622 | 31.69 |
| 32.5468 | 2.7512 | 13.63 |
| 33.3510 | 2.6867 | 10.68 |

61. A process for preparing the salt as in claim 52, comprising: reacting a compound of formula (Ia) with dietylamine, and separating said salt.

62. A process for preparing the salt as in claim 55, comprising: reacting a compound of formula (Ia) with piperazine, and separating said salt.

63. A process for preparing the salt as in claim 58, comprising: reacting a compound of formula (Ia) with 1-(2-hydroxyethyl)pyrrolidine, and separating said salt.
